(19) United States
(12) Patent Application Publication
(10) Pub. No.: US 2022/0089821 A1
(43) Pub. Date: Mar. 24, 2022

(54) HYDROGEL-ENCAPSULATED BETA CELLS, BETA-CELL ENCAPSULATION PROCESS, AND USES THEREOF

(71) Applicant: UNIVERSITY OF WYOMING, Laramie, WY (US)

(72) Inventors: John OAKEY, Laramie, WY (US); Benjamin NOREN, Laramie, WY (US); Zhongliang JIANG, Laramie, WY (US); Kun JIANG, Laramie, WY (US)

(21) Appl. No.: 17/482,143

(22) Filed: Sep. 22, 2021

Related U.S. Application Data
(60) Provisional application No. 63/082,981, filed on Sep. 24, 2020.

(51) Int. Cl. C08J 3/075; C08J 3/28; C08L 33/02; C08L 71/08; C08K 5/37; C12N 5/071

(52) U.S. Cl. CPC C08J 3/075; C08J 3/28; C08L 33/02; C08L 71/08; C08J 2371/08; C12N 5/0676; C08L 2203/02; C08J 2333/02; C08K 5/37

(57) ABSTRACT

Embodiments of the present disclosure generally relate to compositions comprising hydrogel-encapsulated/dispersed beta cells, compositions comprising hydrogel-encapsulated/dispersed beta-cell spheroids, processes for forming such compositions, and uses of the compositions. In an embodiment, a composition is provided that includes a first component comprising a hydrogel, the hydrogel comprising, in polymerized form, one or more photoreactive monomers and a thiol linker. The composition further comprises a second component comprising a plurality of beta cells dispersed or encapsulated within the hydrogel.

### CROSS-REFERENCES TO RELATED APPLICATIONS

This application claims priority to U.S. Provisional Application No. 63/082,981, filed Sep. 24, 2020, which is incorporated herein by reference in its entirety.

### GOVERNMENT RIGHTS

This invention was made with government support under the Faculty Early Career Development Program (BBBE 1254608) awarded by the National Science Foundation and the Wyoming IDeA Networks of Biomedical Research Excellence program (P20GM103432) awarded by the National Institutes of Health. The government has certain rights in the invention.

### BACKGROUND

### Field

Embodiments of the present disclosure generally relate to compositions comprising hydrogel-encapsulated/dispersed beta cells, compositions comprising hydrogel-encapsulated/dispersed beta-cell spheroids, processes for forming such compositions, and uses of the compositions.

### Description of the Related Art

Diabetes is a common metabolic disorder characterized by abnormal blood glucose concentration and the inability to secrete and sense insulin. Type 1 diabetes mellitus (T1DM) is an autoimmune disorder caused by the destruction of beta cells within pancreatic islets. T1DM is commonly treated by insulin therapy though maintaining normal glucose levels with insulin therapy requires several daily injections and monitoring of blood glucose levels. Transplantation of pancreatic islets is another method for treating T1DM, however, the need for life-long immunosuppressive drugs presents a challenge to patients opting for pancreatic islet transplantation. Another method for treating T1DM is transplantation of insulin-secreting pancreatic beta cells within engineered synthetic hydrogels. However, due to, e.g., the extreme vulnerability of beta cells as well as anoikis (programmed cell death), maintaining long-term cell viability within hydrogels remains a significant challenge. One approach to improve the viability of the transplanted beta cells is to transplant beta cell spheroids rather than the beta cells.

Current beta-cell spheroid assembly methods, however, rely heavily on the fabrication of microwell arrays and/or seeding cells to form beta-cell spheroids in individual round-bottomed microwells with one microwell yielding one beta-cell spheroid. Such methods are slow, tedious, exhibit low throughput, and are impractical to produce and harvest the millions of beta-cell spheroids needed to facilitate insulin production in patients presenting diabetes. Further, the produced beta-cell spheroids still show low cell viability, low protection against external deleterious factors as they are targeted by the host's immune system, and low control over insulin generation and glucose sensitivity.

Therefore, there is a need for new compositions and processes to form such compositions comprising hydrogel-encapsulated/dispersed beta cells and to processes for forming such compositions that overcome one or more of these deficiencies.

### SUMMARY

Embodiments of the present disclosure generally relate to compositions comprising hydrogel-encapsulated/dispersed beta cells, compositions comprising hydrogel-encapsulated/dispersed beta-cell spheroids, processes for forming such compositions, and uses of the compositions.

In an embodiment, a composition is provided that includes a first component comprising a hydrogel, the hydrogel comprising, in polymerized form, one or more photoreactive monomers and a thiol linker. The composition further comprises a second component comprising a plurality of beta cells dispersed or encapsulated within the hydrogel.

In another embodiment, a process for forming a composition is provided. The process includes introducing a plurality of beta cells with one or more components to form a reaction mixture, the one or more components comprising a photoreactive monomer, a photoinitiator, a dithiol linker, or combinations thereof. The process further includes introducing a fluorocarbon oil to the reaction mixture, and polymerizing the reaction mixture by exposure to ultraviolet light, under polymerization conditions, to form the composition, the composition comprising the plurality of beta cells dispersed in or encapsulated within a hydrogel.

In another embodiment, a method is provided that includes introducing a composition described herein with a substance that increases insulin secretion or decreases insulin secretion, and monitoring an amount of insulin secretion by at least a portion of the plurality of beta cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 is a schematic of an example device for forming a hydrogel-encapsulated/dispersed cell according to at least one embodiment of the present disclosure.

FIG. 2A is an exemplary image showing example hydrogel-encapsulated/dispersed beta cells according to at least one embodiment of the present disclosure.

FIG. 2B is an exemplary image showing example hydrogel-encapsulated/dispersed beta cells according to at least one embodiment of the present disclosure.

FIG. 3 is a flowchart showing selected operations of an example process for forming hydrogel-encapsulated/dispersed cells according to at least one embodiment of the present disclosure.

FIG. 4A shows images of in-vitro assembled, beta-cell spheroids on culture day 1 formed by a comparative method.

FIG. 4B shows images of in-vitro assembled, beta-cell spheroids on culture day 5 formed by a comparative method.

FIG. 4C is a graph showing the number of cells per well, cell-seeding density, and average spheroid diameter for the in-vitro assembled, beta-cell spheroids formed by a comparative method.

FIG. 5A shows images of the in-vitro assembled, beta-cell spheroids formed by a comparative method.

FIG. 5B is a graph showing the cell viability over 5 days of in-vitro assembled, beta-cell spheroids of different average diameters formed by a comparative method.

FIG. 6A are images of the in-vitro assembled, beta-cell spheroids formed from three beta cells using a comparative method and stained for presence of nuclei (blue), E-cadherin (green), and intracellular insulin (red).

FIGS. 6B-6G are images of in-vitro assembled, beta-cell spheroids of varying sizes formed using a comparative method and stained for presence of nuclei (blue), E-cadherin (green), and intracellular insulin (red).

FIG. 7A is a graph showing exemplary data of the equilibrium swelling ratio as a function of days after polymerization for example hydrogels according to at least one embodiment of the present disclosure.

FIG. 7B is a graph showing exemplary data of the theoretical mesh size as a function of days after polymerization for example hydrogels according to at least one embodiment of the present disclosure.

FIG. 7C is a bar graph showing exemplary data of the elastic modulus for example hydrogels according to at least one embodiment of the present disclosure.

FIG. 8A shows a series of images of example polyethylene glycol norbornene (PEGNB) microgel-encapsulated/dispersed beta cells of varying cell-loading density and varying microgel average diameter on day 1, the microgel formed using a 1500 Dalton (Da) PEG-dithiol linker, according to at least one embodiment of the present disclosure.

FIG. 8B shows a series of images of the example PEGNB microgel-encapsulated/dispersed beta cells of FIG. 8A on day 5 according to at least one embodiment of the present disclosure.

FIG. 8C shows a series of images of example PEGNB microgel-encapsulated/dispersed beta cells of varying cell-loading density and varying microgel average diameter on day 1, the microgel formed using a 3500 Da PEG-dithiol linker, according to at least one embodiment of the present disclosure.

FIG. 8D shows a series of images of the example PEGNB microgel-encapsulated/dispersed beta cells of FIG. 8C on day 5 according to at least one embodiment of the present disclosure.

FIG. 8E is a graph showing exemplary data for the distribution of microgel average diameter of example PEGNB microgel-encapsulated/dispersed beta cells according to at least one embodiment of the present disclosure.

FIG. 8F is a graph showing exemplary data for the cell number per microgel of example PEGNB microgel-encapsulated/dispersed beta cells according to at least one embodiment of the present disclosure.

FIG. 9A shows a series of images of example microgel-encapsulated/dispersed beta cells of varying cell-loading density and varying microgel average diameter on day 1, the hydrogel formed using a 1500 Da PEG-dithiol linker, according to at least one embodiment of the present disclosure.

FIG. 9B shows a series of images of the example microgel-encapsulated/dispersed beta cells of FIG. 9A on day 5 according to at least one embodiment of the present disclosure.

FIG. 9C shows a series of images of example microgel-encapsulated/dispersed beta cells of varying cell-loading density and varying microgel average diameter on day 5, the microgel formed using a 3500 Da PEG-dithiol linker, according to at least one embodiment of the present disclosure.

FIG. 9D shows a series of images of the example microgel-encapsulated/dispersed beta cells of FIG. 9C on day 5 according to at least one embodiment of the present disclosure.

FIG. 10A is a bar graph showing exemplary data of cell viability as a function of cell loading density (15 cells per drop) and microgel average diameter of example microgel-encapsulated/dispersed beta cells, the microgel formed using a 1500 Da PEG-dithiol linker, according to at least one embodiment of the present disclosure.

FIG. 10B is a bar graph showing exemplary data of cell viability as a function of cell loading density (30 cells per drop) and microgel average diameter of example microgel-encapsulated/dispersed beta cells, the microgel formed using a 1500 Da PEG-dithiol linker, according to at least one embodiment of the present disclosure.

FIG. 10C is a bar graph showing exemplary data of cell viability as a function of cell loading density (60 cells per drop) and microgel average diameter of example microgel-encapsulated/dispersed beta cells, the microgel formed using a 1500 Da PEG-dithiol linker, according to at least one embodiment of the present disclosure.

FIG. 10D is a bar graph showing exemplary data of cell viability as a function of cell loading density (15 cells per drop) and microgel average diameter of example microgel-encapsulated/dispersed beta cells, the microgel formed using a 3500 Da PEG-dithiol linker, according to at least one embodiment of the present disclosure.

FIG. 10E is a bar graph showing exemplary data of cell viability as a function of cell loading density (30 cells per drop) and microgel average diameter of example microgel-encapsulated/dispersed beta cells, the microgel formed using a 3500 Da PEG-dithiol linker, according to at least one embodiment of the present disclosure.

FIG. 10F is a bar graph showing exemplary data of cell viability as a function of cell loading density (60 cells per drop) and microgel average diameter of example microgel-encapsulated/dispersed beta cells, the microgel formed using a 3500 Da PEG-dithiol linker, according to at least one embodiment of the present disclosure.

FIG. 11A is a series of exemplary bright field and fluorescent images showing cell viability on day 1 of various amounts of beta cells within microgels (10, 30, or 60 beta cells/microgel), the microgel having an average diameter of 250 μm and made from a 1500 Da PEG-dithiol linker, according to at least one embodiment of the present disclosure.

FIG. 11B is a series of exemplary bright field and fluorescent images showing cell viability on day 5 of the microgels shown in FIG. 11A according to at least one embodiment of the present disclosure.

FIG. 12A is a series of exemplary bright field and fluorescent images showing cell viability on day 1 of various amounts of beta cells within microgels (10, 30, or 60 beta cells/microgel), the microgel having an average diameter of 350 μm and made from a 1500 Da PEG-dithiol linker, according to at least one embodiment of the present disclosure.

FIG. 12B is a series of exemplary bright field and fluorescent images showing cell viability on day 5 of the microgels shown in FIG. 12A according to at least one embodiment of the present disclosure.

FIG. 13A is a series of exemplary bright field and fluorescent images showing cell viability on day 1 of various amounts of beta cells within microgels (10, 30, or 60 beta cells/microgel), the microgel having an average diameter of 450 μm and made from a 1500 Da PEG-dithiol linker, according to at least one embodiment of the present disclosure.

FIG. 13B is a series of exemplary bright field and fluorescent images showing cell viability on day 5 of the microgels shown in FIG. 13A according to at least one embodiment of the present disclosure.

FIG. 14A is a series of exemplary bright field and fluorescent images showing cell viability on day 1 of various amounts of beta cells within microgels (10, 30, or 60 beta cells/microgel), the microgel having an average diameter of 250 μm and made from a 3500 Da PEG-dithiol linker, according to at least one embodiment of the present disclosure.

FIG. 14B is a series of exemplary bright field and fluorescent images showing cell viability on day 5 of the microgels shown in FIG. 14A according to at least one embodiment of the present disclosure.

FIG. 15A is a series of exemplary bright field and fluorescent images showing cell viability on day 1 of various amounts of beta cells within microgels (10, 30, or 60 beta cells/microgel), the microgel having an average diameter of 350 μm and made from a 3500 Da PEG-dithiol linker, according to at least one embodiment of the present disclosure.

FIG. 15B is a series of exemplary bright field and fluorescent images showing cell viability on day 5 of the microgels shown in FIG. 15A according to at least one embodiment of the present disclosure.

FIG. 16A is a series of exemplary bright field and fluorescent images showing cell viability on day 1 of various amounts of beta cells within microgels (10, 30, or 60 beta cells/microgel), the microgel having an average diameter of 450 μm and made from a 3500 Da PEG-dithiol linker, according to at least one embodiment of the present disclosure.

FIG. 16B is a series of exemplary bright field and fluorescent images showing cell viability on day 5 of the microgels shown in FIG. 16A according to at least one embodiment of the present disclosure.

FIG. 17 is an exemplary graph of cell viability as a function of cell-loading density for example microgels made of different thiol linkers according to at least one embodiment of the present disclosure.

FIG. 18A is a series of exemplary images of example microgels encapsulating/dispersing single beta cells (first image of each row) or spheroid-like structures of a range of sizes (remaining images of each row) on day 5 according to at least one embodiment of the present disclosure.

FIG. 18B is a graph showing exemplary data of the viability of beta cells within example microgels of varying average diameters made from a 1500 Da PEG-dithiol linker according to at least one embodiment of the present disclosure.

FIG. 18C is a graph showing exemplary data of the viability of beta cells within example microgels of varying average diameters made from a 3500 Da PEG-dithiol linker according to at least one embodiment of the present disclosure.

FIG. 19A is a series of exemplary immunostaining images of beta cells within example microgels on culture day 5, the microgels formed using a 1500 Da PEG-dithiol linker and having varying cell-loading density and varying microgel average diameter, according to at least one embodiment of the present disclosure.

FIG. 19B is a series of exemplary immunostaining images of beta cells within example microgels on culture day 5, the microgels formed using a 3500 Da PEG-dithiol linker and having varying cell-loading density and varying microgel average diameter, according to at least one embodiment of the present disclosure.

FIG. 20 is a series of exemplary images taken on day 5 of individual and merged color channels showing nuclei, E-cadherin, and intracellular insulin expression of beta cells encapsulated/dispersed within microgels, the microgels having an average diameter of 250 μm and formed using a 1500 Da PEG-dithiol linker, according to at least one embodiment of the present disclosure.

FIG. 21 is a series of exemplary images taken on day 5 of individual and merged color channels showing nuclei, E-cadherin, and intracellular insulin expression of beta cells encapsulated/dispersed within microgels, the microgels having an average diameter of 350 μm and formed using a 1500 Da PEG-dithiol linker, according to at least one embodiment of the present disclosure.

FIG. 22 is a series of exemplary images taken on day 5 of individual and merged color channels showing nuclei, E-cadherin, and intracellular insulin expression of beta cells encapsulated/dispersed within microgels, the microgels having an average diameter of 450 μm and formed using a 1500 Da PEG-dithiol linker, according to at least one embodiment of the present disclosure.

FIG. 23 is a series of exemplary images taken on day 5 of individual and merged color channels showing nuclei, E-cadherin, and intracellular insulin expression of beta cells encapsulated/dispersed within microgels, the microgels having an average diameter of 250 μm and formed using a 3500 Da PEG-dithiol linker, according to at least one embodiment of the present disclosure.

FIG. 24 is a series of exemplary images taken on day 5 of individual and merged color channels showing nuclei, E-cadherin, and intracellular insulin expression of beta cells encapsulated/dispersed within microgels, the microgels having an average diameter of 350 μm and formed using a 3500 Da PEG-dithiol linker, according to at least one embodiment of the present disclosure.

FIG. 25 is a series of exemplary images taken on day 5 of individual and merged color channels showing nuclei, E-cadherin, and intracellular insulin expression of beta cells encapsulated/dispersed within microgels, the microgels having an average diameter of 450 μm and formed using a 3500 Da PEG-dithiol linker, according to at least one embodiment of the present disclosure.

FIG. 26A is a bar graph showing exemplary data (on day 1) of insulin secretion in response to glucose stimulation from example beta-cell-laden microgels made from a 1500 Da PEG-dithiol linker or a 3500 Da PEG-dithiol linker according to at least one embodiment of the present disclosure.

FIG. 26B is a bar graph showing exemplary data (on day 5) of insulin secretion in response to glucose stimulation from the example beta-cell-laden microgels of FIG. 26A according to at least one embodiment of the present disclosure.

Figures included herein illustrate various embodiments of the disclosure. It is contemplated that elements and features of one embodiment may be beneficially incorporated in other embodiments without further recitation.

### DETAILED DESCRIPTION

Embodiments of the present disclosure generally relate to compositions comprising hydrogel-encapsulated/dispersed beta cells, compositions comprising hydrogel-encapsulated/dispersed beta-cell spheroids, processes for forming such compositions, and uses of the compositions. The compositions can be used for, e.g., therapeutic applications and as tools for drug screening and drug discovery. Briefly, embodiments of the compositions include a hydrogel formed from the polymerization of photoreactive monomers. A beta cell or a plurality of beta cells can be encapsulated, dispersed, suspended, retained, or otherwise held in the hydrogel. The inventors have found that these compositions can, e.g., enhance survival of the beta cells, improve retention of the beta cells, control delivery of the beta cells, and control gene expression of therapeutic beta cells relative to conventional techniques. Further, the inventors found that the compositions described can facilitate assembly of beta-cell spheroids from individual beta cells in a manner that surpasses conventional methods of assembling beta-cell spheroids. Here, the compositions described herein show an increased number of beta-cell spheroids assembled in a given timespan relative to conventional methods such as microwell-based assembly. Moreover, the processes for forming the compositions which facilitate the formation of beta-cell spheroids is, e.g., significantly easier in terms of production and collection as well as lower in cost than conventional methods.

In some examples, processes described herein can generally include introducing a plurality of beta cells (e.g., two or more beta cells), one or more polymerizable monomers, and an oil (e.g., a fluorocarbon oil) to a microfluidic device. Due to physical interactions between the oil and the other components introduced to the microfluidic device, droplets having the beta cells and polymerizable species therein are formed. The droplets containing the cells and polymerizable monomers are then exposed to ultraviolet (UV) light as they travel through the microfluidic device. The UV light polymerizes the one or more polymerizable monomers into a cross-linked hydrogel network encapsulating/dispersing the beta cells in, e.g., microscopic hydrogels. If desired, hydrogels containing the beta cells can be isolated and re-suspended for use in, e.g., therapeutic applications including injection and topical administration. Embodiments of the processes described herein enable the creation of tunable, biocompatible microenvironments suitable for encapsulation and/or dispersion of living pancreatic beta cells in sufficient quantities to enable their development into functional spheroid or spheroid-like structures within the hydrogel microparticle.

Patients with type 1 diabetes typically suffer from insulin deficiency due to the dysfunction of pancreatic beta cells. Despite the inconvenience and cost, insulin injections two to five times per day is the most common management practice for type 1 diabetes. Nevertheless, hyperglycemia and hypoglycemia can frequently occur in patients due to the insensitivity of injection therapies to regulate blood glucose levels. As an alternative route to achieve insulin independence and to regulate blood glucose levels, insulin-secreting pancreatic beta cells capable of dynamically regulating glucose levels have been considered. To avoid immunogenicity-induced early termination of the exogenous beta cells, and also to provide the exogenous beta cells with a physiologically relevant environment, semi-permeable hydrogels have been used to encapsulate and isolate implanted beta cells from the patient's immune cells. However, much of the focus of hydrogels is on macroscopic hydrogels in which cell distribution was randomized and cells in the center of the hydrogel fell out of the efficient diffusional length scale for the transportation of physiology-relevant small molecules like, oxygen and nutrients. In addition, the translation of hydrogels to clinical practice have been constrained by the requirement of surgical implantation of the hydrogel.

Miniaturization techniques to produce injectable microgels that avoid the risks and costs associated with surgery have been investigated. Conventional fabrication methods to produce injectable microgels, such as stop-flow lithography (SFL), continuous-flow lithography (CFL), and bioprinting have been used to fabricate cell-laden microgels with tunable hydrogel properties. However, such methods exhibit low fabrication throughput.

Regardless of the hydrogel-fabrication method, the materials employed to make the hydrogels, the polymerization scheme utilized, and the dimensions of the resulting hydrogels, maintaining long-term beta-cell viability within hydrogels remains a challenge. The challenge is due to, e.g., the extreme vulnerability of beta cells as well as the lack of cell-cell interactions and/or cell-matrix interactions when the beta cells are encapsulated/dispersed in hydrogels. In vitro beta-cell spheroid assembly has been achieved by seeding single beta cells into microwells, and the beta cells within an individual microwell would start to initiate contact with each other and eventually form cell clusters. Traditionally-used microwells, such as flat-bottom microwells, are capable of forming large beta-cell spheroids (200 μm) with a large quantity of beta cells. However, all studies using microwells to form beta-cell spheroids have failed to address the quantitative requirement for beta-cell spheroid assembly, a fundamental question governing beta-cell spheroids assembly principles. Additionally, throughput using microwells is too low for functionality analyses and clinical studies as the efficiency of forming beta-cell spheroids within microwells relies heavily on the time consumed on microwell fabrication, cell-seeding, and beta-cell spheroid recovery, which all exhibit very low throughput and cumbersome processes. The lack of knowledge regarding beta-cell spheroid requirements, particularly the minimum number of beta cells necessary to form a specific cellular structure, and the effects of beta-cell spheroid size on cellular tolerance has limited the potential of such applications.

These aforementioned issues, as well as others, are addressed by embodiments described herein. As described herein, embodiments of the compositions and processes enable the high-throughput of beta cells and beta-cell spheroid assembly within hydrogels, thereby addressing various fundamental fabrication challenges and key bottlenecks in manufacturing viable beta cells the creation of, e.g., ‘artificial pancreas’ treatment for type 1 diabetes.

The hydrogels described herein can be of various suitable sizes, shapes, and/or morphologies. While the present disclosure refers to “microgels”, “microspheres”, “microcapsules”, and “microparticles”, it will be appreciated that the disclosure may be applied to gels, spheres, capsules, and particles having a smaller size (e.g., “nanogels”, “nanospheres”, “nanocapsules”, or “nanoparticles”) or gels, spheres, capsules, and particles having a larger size (e.g., “macrogels”, “macrospheres”, “macrocapsules”, or “macroparticles”). The hydrogels can be in the form of droplets. The terms “gels”, “spheres”, “capsules”, “particles”, and “droplets” are used interchangeably unless the context clearly indicates otherwise. For example, the term “microgels” refers to microgels, microspheres, microcapsules, and microparticles unless the context clearly indicates otherwise. In addition, the terms “spheroid” and “spheroid-like” are used interchangeably unless the context clearly indicates otherwise. For example, beta-cell spheroid structures refers to both beta-cell spheroid structures and beta-cell spheroid-like structures.

Also, while embodiments and examples are described herein with reference to hydrogel encapsulation of beta cells and/or beta-cell spheroids, it is contemplated that the beta cells beta-cell spheroids can additionally, or alternatively, be suspended, dispersed, retained, or otherwise held in the hydrogels. For example, the microfluidic device described herein can be utilized to form hydrogels having beta cells and/or beta-cell spheroids dispersed therein, and embodiments of processes for forming the hydrogel-encapsulated beta cells and/or beta-cell spheroids can be used to form hydrogels having beta cells and/or beta-cell spheroids dispersed therein.

As described above, maintaining the long-term survivability of beta cells remains a challenge due to the extreme vulnerability of beta cells and anoikis—programmed cell death induced by inadequate or inappropriate cell-matrix. To overcome these and other issues, embodiments described herein can facilitate formation of beta-cell spheroids. Beta-cell spheroids are clusters or aggregates of two or more beta cells when the beta cells contact or touch. Such cell-cell contact between beta cells is important for maintaining survival of the beta cells and normal insulin secretion from the beta cells.

Various methods have been developed to promote cell-cell contact between beta cells, however, these methods are, e.g., expensive, lack the ability for large-scale production, and/or do not maintain beta cell viability. For example, microwell cell-culture platforms are widely utilized to aggregate beta cells so that the beta cells can form beta-cell spheroids. Fabrication of such microwells or microwell arrays, however, is costly for at least the reason that the dimensions of the cell-culture wells are not easily tunable. In contrast, embodiments described herein enable, e.g., the easy tunability of hydrogel dimensions by, for example, changing the materials used to form the hydrogels, the polymerization conditions utilized, among other variables. By being able to tune the dimensions of the hydrogel, a user or manufacturer can easily adjust the amount of beta cells in/within the hydrogel matrix. The material properties of the hydrogel are also easily tunable by embodiments described herein. For example, selection of, e.g., the photoreactive monomers (e.g., monomer type/class, monomer size), the thiol linkers (type and size), and/or polymerization conditions, among other conditions, enables easy adjustment of and control over, e.g., the degradation properties of the hydrogels, the amount of beta cells encapsulated/dispersed, et cetera. Changing the photoreactive monomers, thiol linkers, and/or polymerization conditions, can only entail changing the hydrogel forming solution used to form the hydrogel that encapsulates/disperses the beta cells. Moreover, embodiments described herein enable control over the beta-cell cluster size. Further, the materials utilized for the hydrogels enable promotion of cell-cell interaction over cell-material interaction, thereby mitigating cell death.

Assembly of the beta-cell spheroids and beta-cell spheroid-like structures enabled by embodiments described herein can mimic the function(s) of the body's natural glucose-controllers, e.g., the insulin-secreting beta cells of the pancreas. As such, embodiments described herein can enable creation of an artificial pancreas. Moreover, the compositions, and processes for forming such compositions, described herein can provide a high-throughput route to transplantable beta-cell spheroids for the treatment of diabetes. Further, the compositions described herein show, e.g., an enhanced ability to control insulin generation in response to glucose relative to conventional compositions.

Also described herein are uses of the compositions comprising hydrogel-encapsulated/dispersed beta cells and/or beta-cell spheroids. Such uses include therapeutic applications for, e.g., the treatment of diabetes. Other uses can include utilization of the compositions in a drug-discovery pipeline. For therapeutic applications as well as drug-screening, large amounts of beta-cell spheroids are needed. However, conventional methods of forming beta-cell spheroids are, e.g., very slow, have low throughput, and are impractical for producing and harvesting the millions of beta-cell spheroids needed to facilitate insulin production in patients presenting diabetes as well as for drug screening. In contrast, the processes described herein significantly improves on the number of beta-cell spheroids assembled in a given timespan, as well as their ease of production and collection. The processes described herein also enable compositions having an increased duration of beta-cell viability and enhanced control over insulin generation and glucose sensitivity compared to current state-of-the-art methods. In addition, the hydrogels can protect the beta cells against external deleterious factors, and can show controlled degradation rates, based on, e.g., chemical and material properties of the hydrogel material. Such degradation rates can be factors in regulating beta-cell spheroid assembly and play a role in the glucose sensitivity of the encapsulated/dispersed beta cells.

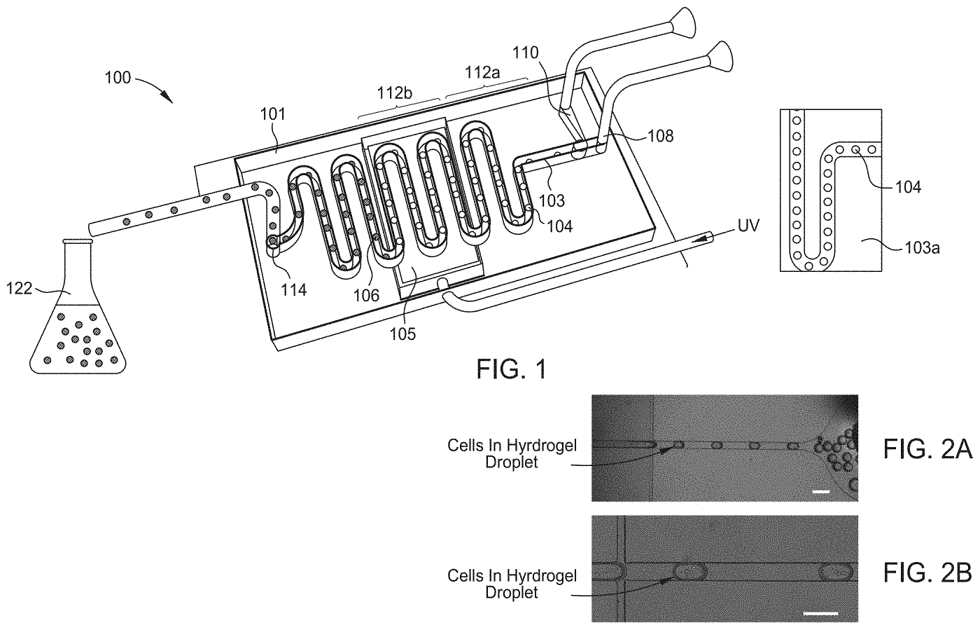

FIG. 1 is a schematic of an example device 100 for forming hydrogel-encapsulated/dispersed beta cells according to at least one embodiment of the present disclosure. Such hydrogel-encapsulated/dispersed beta cells produced can be in the form of microparticles. Device 100 can be used for continuous production of hydrogel-encapsulated/dispersed beta cells.

Device 100 includes a microfluidic device 101 having a fluidic channel 103. In at least one embodiment, the fluidic channel 103 has a diameter of micrometers (μm) to millimeters (mm). For example, the fluidic channel 103 has a diameter from about 1 μm to about 2 mm and/or a depth of about 1 μm to about 2 mm. One or more portions of the fluidic channel 103 can be in the form of loops, discussed below. The fluidic channel 103 includes a mixing area 112a where a hydrogel forming solution, discussed below, can be mixed with beta cells and oil, and a polymerization area 112b where monomers of the hydrogel forming solution polymerize to form hydrogels that encapsulate and/or disperse the beta cells.

As stated above, portions of the fluidic channel 103 can be in the form of loops. The loops enable control over, e.g., the kinetics of mixing, the kinetics of polymerization, the exposure time for polymerization, and/or the gelation of the hydrogels. That is, the loops can enable uniform processing of microparticles. Other morphologies or shapes besides, or in addition to, loops are contemplated to enable processing of the microparticles. Such morphologies or shapes include spirals or other tortuous paths. That is, any suitable morphology or shape that extends the length of the fluidic channel 103 in, e.g., the mixing area 112a and/or the polymerization area 112b would have the same or similar effect of controlling the exposure time so that the desired cross-linking can be achieved on a microfluidic chip with high-throughput droplet production capabilities.

The microfluidic device 101 has an opening 110 for introducing a hydrogel forming solution to the fluidic channel 103. The hydrogel forming solution includes photoinitiators, reaction components, and/or photoreactive monomers (e.g., PEG-dithiol linker, PEGNB, PEGDA, PLA, etc.). Beta cells in, e.g., a buffer, can be introduced to the fluidic channel 103 via opening 110 or a separate opening. The microfluidic device 101 includes another opening 108 for introducing a suspension fluid to the fluidic channel 103. The suspension fluid can be an oil, such as a fluorocarbon oil. The oil can serve to pinch off the beta cells and hydrogel forming solution (e.g., photoinitiators, reaction components, and/or photoreactive monomers) into droplets and carry the droplets through the microfluidic device 101. Openings 108 and 110 are coupled to the fluidic channel 103. As shown, tubings are coupled to the individual openings 108, 110 to allow introduction of the oil, beta cells, hydrogel forming solution, and/or other reaction components to the fluidic channel 103 of the microfluidic device 101. However, it is contemplated that introduction of the oil, beta cells, hydrogel forming solution, and/or other reaction components to the microfluidic device 101 can be performed in other suitable ways, such as direct connecting Leuer lock type devices, snap-together microfluidic assemblies, and syringe-like devices, without departing from the scope of the present disclosure.

Although two openings are described, more or less openings can be used to introduce the oil, beta cells, hydrogel forming solution, and/or other reaction components to the microfluidic device 101. The inset identified as 103a is a pictorial representation of the fluidic channel 103 showing droplets 104 in suspension fluid (e.g., the oil). The droplets 104 can include, but are not limited to, beta cells, photoreactive monomers, photoinitiators, reaction components, and/or fluorocarbon oil, as well as other materials.

The fluidic channel 103 includes the polymerization area 112b. At the polymerization area 112b, monomers and/or reaction components of the droplets 104 polymerize to form, e.g., a hydrogel 106, that suspends, disperses, encapsulates, retains, or otherwise holds a beta cell or a plurality of beta cells. As shown, the fluidic channel 103 of the polymerization area 112b includes a suitable number of loops (and/or other suitable shape) to enable, e.g., sufficient polymerization of the monomers and other reaction components as well as sufficient gelation of the hydrogels.

The device 100 further includes a polymerization control device 105 optically and/or mechanically coupled to at least a portion of the fluidic channel 103. The polymerization control device 105 is configured to cause a polymerization reaction when the desired materials are within the polymerization area 112b. The polymerization control device 105 can include a UV-light source(s), such as a UV lamp, UV light source concentrated via lenses and/or microscope objective, or laser, that polymerizes the monomers and/or reaction components to form the hydrogel (e.g., hydrogels 106). Coupling of the polymerization control device 105 can take multiple forms. For example, the microfluidic device 101 can be placed on top of, below, or otherwise adjacent to, the polymerization control device 105. The UV light source can be located in a stand-alone unit outside of the microfluidic device 101.

FIGS. 2A and 2B are exemplary images of the polymerized hydrogels within the fluidic channel 103 of the polymerization area 112b. A portion of the image shows beta cells in a hydrogel droplet. After polymerization, the cell-laden microparticles (e.g., the hydrogel-encapsulated/dispersed beta cells) move toward the fluidic channel exit 114 where the cell-laden microparticles can be collected via any suitable collection unit 122, e.g., flask, centrifuge tube, reservoir, vessel, or the like. Other materials (byproducts, suspension fluid, unreacted materials, etc.) can exit the fluidic channel exit 114 along with the hydrogel-encapsulated/dispersed beta cells. Accordingly, the hydrogel-encapsulated/dispersed beta cells or compositions comprising the hydrogel encapsulated/dispersed beta cells can be purified, or otherwise isolated, from the other materials exiting the microfluidic device 101.

Movement of the various materials (e.g., suspension fluid, and beta cells, photoreactive monomers, photoinitiators, and/or reaction components, etc.) from the one or more openings 108, 110 to the fluidic channel exit 114 can be controlled by, e.g., capillary action, laminar flow, temperature, a pumping mechanism (e.g., a syringe pump, pressure pump, or piezoelectric pump), electrodes, and the like. Such elements controlling the movement can be placed at either opposing ends of the device, opposite ends, or along various regions along a length of the fluidic channel 103.

As discussed above, the photoreactive monomers used to form the hydrogel contain photoreactive functional groups chemically attached to, e.g., polyethylene glycol (PEG). Illustrative, but non-limiting, examples of photoreactive functional groups include alkenes, thiols, acids, or combinations thereof. Upon irradiation, the photoreactive monomers (with or without co-reactants, such as linkers described below) polymerize to form a hydrogel.

Non-limiting examples of photoreactive monomers include, but are not limited to, polyethylene glycol norbornene (PEGNB), polyethylene glycol diacrylate (PEGDA), derivatives thereof, or combinations thereof. The photoreactive monomers can be branched (e.g., ˜20 k 4-arm PEGNB and ˜40 k 8-arm PEGNB) or unbranched. Other PEG-based derivatives having varied reactive functional groups are also contemplated. The molecular weight and shape (e.g., number of arms on PEGNB) of one or more photoreactive monomers, among other characteristics, can be changed. Changing the molecular weight and shape of the photoreactive monomers (as well as the linker) can enable the tuning of various properties of the hydrogel polymer matrix and can confer a range of traits to the system depending on desired use and desired effect on encapsulated/dispersed cellular function.

Photoreactive monomers can also include non-PEG-based monomers such as acrylates, acids (e.g., lactic acid, hyaluronic acid), gelatin, collagen, or combinations thereof. For example, polylactic acid (PLA) and derivatives thereof can be used. Block copolymers and triblock copolymers can also be used such as triblock PLA and PLA-PEG-PLA.

Molecular conformation of the photoreactive monomers can be varied to, e.g., impart desired material properties to the hydrogel microenvironment. For example, 1-arm molecular structures to 12-arm molecular structures can be used, such as 4-arm, 8-arm, or 12-arm molecular structures, such as 4-arm PEGNB, 8-arm PEGNB, 12-arm PEGNB, or combinations thereof. Further, the chemical properties of the hydrogel microenvironment can be modified via click chemistry through addition of thiolated agents (for, e.g., PEGNB) or similar acrylated agents (for, e.g., PEGDA) such as thiolated or acrylated cell adhesion peptides like RGD (arginine-glycine-aspartate) or CRGDS (cystine-arginine-glycine-aspartate-serine). Mixtures of one or more photoreactive monomers, e.g., a mixture of PEGNB and PEGDA) can also be used, as well as mixtures that include non-PEG-based photolabile hydrogels such as gelatin methacrylate and/or photolabile hyaluronic acid.

A molecular weight of the one or more photoreactive monomers can be from about 250 Da to about 50,000 Da, such as from about 5,000 Da to about 50,000 Da, such as from about 10,000 Da to about 45,000 Da, such as from about 15,000 Da to about 40,000 Da, such as from about 20,000 Da to about 35,000 Da, such as from about 25,000 Da to about 30,000 Da. Illustrative, but non-limiting, examples of the molecular weight of the photoreactive monomer are from about 250 Da to about 10,000 Da, such as from about 500 Da to about 9,000 Da, such as from about 1,000 Da to about 8,000 Da, such as from about 2,000 Da to about 7,000 Da, such as from about 3,000 Da to about 6,000 Da, such as from about 4,000 Da to about 5,000 Da. In some examples, the molecular weight of the one or more photoreactive monomers is 30,000 Da or less. Higher or lower molecular weights of the one or more photoreactive monomers are contemplated. The molecular weight of the photoreactive monomer refers to the number average molecular weight (Mₙ). The Mₙ is the Mₙ provided by the manufacturer of the photoreactive monomer.

The photoreactive monomers can be introduced to the microfluidic device 101 in the form of a hydrogel forming solution. The hydrogel forming solution can contain one or more photoreactive monomers, one or more photoinitiators, one or more linkers, one or more cell adhesion peptides, or combinations thereof, as well as additional components. Suitable organic and/or aqueous solvents are utilized as a portion of the hydrogel forming solution. Such organic and/or aqueous solvents can include water, saline, phosphate buffered saline, appropriate biologically compatible liquid, or combinations thereof.

A concentration of the one or more photoreactive monomers useful for the hydrogel forming solution can be from about 5 wt % to about 75 wt %, such as from about 10 wt % to about 70 wt %, such as from about 15 wt % to about 65 wt %, such as from about 20 wt % to about 60 wt %, such as from about 25 wt % to about 55 wt %, such as from about 30 wt % to about 50 wt %, such as from about 35 wt % to about 45 wt %, based on a total weight percent of the components of the hydrogel forming solution (not to exceed 100 wt %). In at least one embodiment, the concentration of the one or more photoreactive monomers in the hydrogel forming solution is from about 5 wt % to about 35 wt %, such as from about 10 wt % to about 30 wt %, such as from about 15 wt % to about 25 wt %, based on the total weight percent of the components of the hydrogel forming solution (not to exceed 100 wt %). Higher or lower concentrations of the one or more photoreactive monomers can be used depending on application.

The components that are subjected to polymerization can further include one or more linkers, such as a dithiol linker, such as a polyethylene glycol-dithiol (PEG-dithiol) linker, a derivative thereof, or combinations thereof. PEG-dithiol is a thiolated PEG having two thiol groups. The linker can be referred to as a thiol-containing monomer or dithiol linker unless the context indicates otherwise. When a dithiol linker is utilized, the photoreactive monomer(s) polymerize with the thiol-containing monomer(s) via a step-growth polymerization reaction occurring between the ene portion of the monomers and the thiol of the thiol-containing monomer.

A molecular weight of the one or more linkers (e.g., the PEG-dithiol linker) can be from about 500 Da to about 10,000 Da, such as from about 1,000 Da to about 9,500 Da, such as from about 1,500 Da to about 9,000 Da, such as from about 2,000 Da to about 8,500 Da, such as from about 2,500 Da to about 8,000 Da, such as from about 3,000 Da to about 7,500 Da, such as from about 3,500 Da to about 7,000 Da, such as from about 4,000 Da to about 6,500 Da, such as from about 4,500 Da to about 6,000 Da, such as from about 5,000 Da to about 5,500 Da. In some examples, the molecular weight of the linker is about 6,000 Da or less, such as from about 500 Da to about 6,000 Da, such as from about 1,000 Da to about 5,000 Da, such as from about 1,500 Da to about 4,500 Da, such as from about 2,000 Da to about 4,000 Da, such as from about 2,500 Da to about 3,500 Da. The molecular weight of the linker refers to the number average molecular weight (Mₙ). The Mₙ is the Mₙ provided by the manufacturer of the linker. Higher or lower molecular weights of the one or more linkers are contemplated. Illustrative, but non-limiting, examples of PEG-dithiol linkers include ˜1.5 k PEG-dithiol, 3.5 k PEG-dithiol, and ˜5 k PEG-dithiol.

A concentration of the one or more linkers (e.g., PEG-dithiol) in the hydrogel forming solution can be from about 1 mM to about 50 mM, such as from about 5 mM to about 45 mM, such as from about 10 mM to about 40 mM, such as from about 15 mM to about 35 mM, such as from about 20 mM to about 30 mM, based on a total molar concentration of the components of the hydrogel forming solution. Higher or lower concentrations of the one or more linkers can be used depending on application.

The hydrogel forming solution can also include one or more photoinitiators. Illustrative, but non-limiting, examples of photoinitiators include lithium phenyl-2,4,6-trimethylbenzoylphosphinate (LAP) photoinitiator, 2-hydroxy-2-methyl propiophenone (e.g., Irgacure™ 1173, Darocur™ 1173), and combinations thereof. A concentration of the one or more photoinitiators in the hydrogel forming solution can be from about 0.0001 wt % to about 1 wt %, such as from about 0.001 wt % to about 0.9 wt %, such as from about 0.01 wt % to about 0.5 wt %, such as from about 0.05 wt % to about 0.1 wt %, based on the total wt % of the components of the hydrogel forming solution. Higher or lower concentrations of the one or more photoinitiators can be used depending on, e.g., the application or desired results.

The chemical properties of the hydrogel microenvironment can be modified via click chemistry through addition of thiolated agents such as thiolated cell adhesion peptides like RGD or CRGDS. In some embodiments, the hydrogel forming solution can include one or more cell adhesion peptides such as RGD, CRGDS, or a combination thereof. A concentration of the one or more cell adhesion peptides in the hydrogel forming solution can be from about 0.5 mM to about 10 mM, such as from about 1 mM to about 8 mM, such as from about 2 mM to about 6 mM, such as from about 3 mM to about 4 mM based on the total molar concentration of the components of the hydrogel forming solution.

Beta cells in a suitable media such as an aqueous buffer Dulbecco's Modified Eagle's Medium (DMEM), such as phosphate buffered saline, are also introduced to the microfluidic device 101. The beta cells in media can be part of the hydrogel forming solution. A concentration of beta cells in the suitable media or in the hydrogel forming solution that are introduced or otherwise delivered to the microfluidic device 101 can be from about 1 cell/mL to about 1×10⁹ cells/mL, such as from about 1×10³ cells/mL to about 1×10⁸ cells/mL, such as from about 1×10⁵ cells/mL to about 1×10⁷ cells/mL. A higher or lower concentration of beta cells in the suitable media or in the hydrogel forming solution can be utilized.

Additional reaction components such as reaction mixture precursors, solvents, catalysts, reagents, and the like, can be introduced to the microfluidic device 101. These additional reaction components can mix and/or interact (e.g., chemically and/or physically) with the components of the hydrogel forming solution, and/or the oil to form the hydrogel-encapsulated beta cells.

Using the components described above, various formulations can be used to form the hydrogel-encapsulated/dispersed beta cells, hydrogel-encapsulated/dispersed beta-cell spheroids, combinations thereof, or compositions thereof. The formulation can be that of the hydrogel forming solution or separate solutions that are introduced to the microfluidic device or other suitable devices to form hydrogels.

A non-limiting formulation useful for the polymerization can include (a) from about 0.1 wt % to about 40 wt %, such as from about 1 wt % to about 40 wt %, such as from about 5 wt % to about 35 wt %, such as from about 10 wt % to about 20 wt % of one or more photoreactive monomers, such as a PEGNB, ranging in molecular weight from about 500 Da to about 50,000 Da, such as from about 3,000 Da to about 50,000 Da, such as from about 5,000 Da to about 20,000 Da, such as from about 10,000 Da to about 15,000 Da; (b) from about 1 mM to about 100 mM, such as from about 5 mM to about 50 mM PEG dithiol ranging in molecular weight from about 100 Da to about 10,000 Da; and/or (c) from about 0.0001 wt % to about 1 wt %, such as from about 0.01 wt % to about 0.1 wt % of LAP photoinitiator. Additional components can be used as desired.

When PEGNB is utilized with a second photoreactive monomer such as PEGDA, PLA, PLA-PEG-PLA, etc., a non-limiting formulation can include the aforementioned formulation with about 0.1 wt % to about 40 wt %, such as from about 1 wt % to about 40 wt %, such as from about 5 wt % to about 35 wt %, such as from about 10 wt % to about 20 wt % of the second photoreactive monomer (e.g., PEGDA, PLA, PLA-PEG-PLA, etc.) having a molecular weight from about 1,000 Da to about 30,000 Da, such as from about 5,000 Da to about 20,000 Da, such as from about 10,000 Da to about 15,000 Da. Additional components can be used as desired.

An illustrative, but non-limiting, formulation useful to form a PEGPLA/NB composite hydrogels can include: (a) from about 0.1 wt % to about 40 wt %, such as from about 1 wt % to about 40 wt %, such as from about 5 wt % to about 35 wt % such as from about 10 wt % to about 20 wt % of a first photoreactive monomer (e.g., PLA-PEG-PLA, etc.) having a molecular weight from about 1,000 Da to about 30,000 Da, such as from about 5,000 Da to about 20,000 Da, such as from about 10,000 Da to about 15,000 Da; (b) from about 0.1 wt % to about 40 wt %, such as from about 1 wt % to about 40 wt %, such as from about 5 wt % to about 35 wt %, such as from about 10 wt % to about 20 wt % of a second photoreactive monomer (e.g., PEGNB, such as 4-arm PEGNB, 8-arm PEGNB, or a combination thereof) ranging in molecular weight from about 500 Da to about 50,000 Da, such as from about 3,000 Da to about 50,000 Da, such as from about 5,000 Da to about 20,000 Da, such as from about 10,000 Da to about 15,000 Da; (c) from about 1 mM to about 100 mM, such as from about 5 mM to about 50 mM PEG dithiol ranging in molecular weight from about 100 Da to about 10,000 Da; and/or (d) from about 0.0001 wt % to about 1 wt %, such as from about 0.01 wt % to about 0.1 wt % of the LAP photoinitiator.

In some examples, a concentration of beta cells used for the hydrogel forming solution is from about 1×10⁴ cells/mL to about 1×10⁹ cells/mL, such as from about 1×10⁵ cells/mL to about 1×10⁸ cells/mL, 1×10⁶ cells/mL to about 1×10⁷ cells/mL.

Embodiments of the present disclosure also generally relate to processes for forming compositions that include a plurality of beta cells (e.g., two or more beta cells) encapsulated, dispersed, suspended, retained, or otherwise held in a hydrogel. The beta cells of the hydrogel-encapsulated/dispersed beta cells or compositions thereof can be in the form of beta-cell spheroids or beta-cell spheroid-like structures. The spheroid or spheroid-like morphology of the beta cells is indicative of a grouping of a plurality of beta cells (e.g., two or more beta cells) that contact, touch, or otherwise aggregate. Briefly, and in some examples, the process generally includes forming a reaction mixture that includes beta cells and one or more photoreactive monomers, and then polymerizing the reaction mixture to form the hydrogel-encapsulated/dispersed beta cells or compositions comprising hydrogel-encapsulated/dispersed beta cells. In some embodiments, processes of forming hydrogel-encapsulated/dispersed beta cells include forming droplets, having beta cells and polymerizable species therein, within an oil in a microfluidic device. In contrast to conventional techniques of forming beta-cell spheroids, processes described herein can enable the rapid formation of beta-cell spheroids as the hydrogel material can be tuned based on, e.g., physical or chemical properties. Moreover, the resulting beta-cell spheroids and/or spheroid-like structures show, e.g., an enhanced ability to control insulin generation in response to glucose relative to conventional processes for forming beta-cell spheroids.

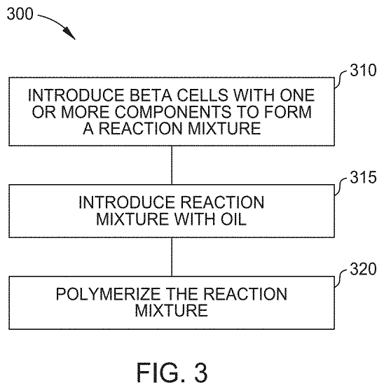

FIG. 3 is a flowchart showing selected operations of an example process 300 for forming hydrogel-encapsulated/dispersed beta cells or compositions comprising hydrogel-encapsulated/dispersed beta cells. Process 300 can be performed in a microfluidic device such as the microfluidic device 101. However, it is contemplated that any suitable device or tool can be used to form the hydrogel-encapsulated/dispersed beta cells or compositions comprising hydrogel-encapsulated/dispersed beta cells such as a droplet generator, emulsion, or other microfluidic device.

Process 300 begins at operation 310 with introducing beta cells (e.g., a plurality of beta cells, e.g., 2 or more beta cells) with one or more components in, e.g., the microfluidic device 101, to form a reaction mixture. The beta cells can be in the form of a suspension in an aqueous buffer such as phosphate buffered saline (PBS). The one or more components can include one or more photoreactive monomers, one or more linkers (e.g., dithiol linker), one or more photoinitiators, and/or one or more solvents. Other materials such as reagents, catalysts, and/or cell-adhesion peptides can be optionally added. The reaction mixture can be in the form of microparticles in solution in the presence of the oil. These microparticles can be created in sizes ranging from, e.g., about 1 μm to about 2000 μm, such as from about 2 μm to about 1000 μm, such as from about 4 μm to about 500 μm, with beta cell concentrations ranging from, e.g., 1 beta cell per microparticle to thousands of beta cells per microparticle, or more.

Operation 310 can include flowing a hydrogel forming solution into the microfluidic device 101 at a flow rate of about 0.1 μL/min to about 150 μL/min, such as from about 25 μL/min to about 125 μL/min, such as from about 50 μL/min to about 100 μL/min, such as from about 80 μL/min to about 100 μL/min. Higher or lower flow rates are contemplated for the hydrogel forming solution. In some embodiments, and when the hydrogel forming solution does not include beta cells, operation 310 can further include flowing a beta-cell stream—a plurality of beta cells in a suspension or suitable media such as a buffer, such as PBS—into the microfluidic device 101 at a flow rate of about 0.1 μL/min to about 150 μL/min, such as from about 25 μL/min to about 125 μL/min, such as from about 50 μL/min to about 100 μL/min, such as from about 80 μL/min to about 100 μL/min. Higher or lower flow rates are contemplated for this beta-cell stream. In some embodiments, the hydrogel forming solution and the cell-stream are introduced at the same time or separate times to the same or different opening of the microfluidic device.

At operation 315, an oil (e.g., a fluorocarbon oil) can be introduced to the reaction mixture. Upon introduction, the oil with the reaction mixture can form droplets. Here, for example, the oil is added to the microfluidic device 101, and the oil can aid in the formation of droplets within the fluidic channel. Such droplets help, e.g., bring together the polymerizable reactants and the beta cells. A flow rate of the oil into the microfluidic device 101 can be from about 0.1 μL/min to about 200 μL/min, such as from about 1 μL/min to about 150 μL/min, such as from about 25 μL/min to about 125 μL/min, such as from about 50 μL/min to about 100 μL/min, such as from about 80 μL/min to about 100 μL/min. Higher or lower flow rates are contemplated for this oil stream.

In some examples, the flow rate of the hydrogel forming solution ranges from about 0.5 μL/min to 100 μL/min and/or the flow rate of the oil ranges from about 2 μL/min to about 300 μL/min

The process 300 further includes polymerizing the reaction mixture to form a hydrogel-encapsulated/dispersed beta cells, or compositions thereof, at operation 320. The polymerization reaction of operation 320 can be performed under polymerization conditions. Polymerization of the reaction mixture forms the hydrogel-encapsulated/dispersed beta cells and/or compositions comprising hydrogel-encapsulated/dispersed beta cells. The beta cells can be in the form of beta cells, beta-cell spheroids, beta-cell spheroid-like structures, or combinations thereof. In some embodiments, the pH of the reaction mixture before, during, and/or after polymerization can be from about 5 to about 9, such as from about 6 to about 8, such as from about 6.5 to about 7.5.

Polymerization conditions can include exposing the reaction mixture to UV light at a desired wavelength or wavelength range, such as a wavelength or wavelength range of about 290 nm to about 500 nm, such as from about 320 nm to about 460 nm, such as from about 340 nm to about 440 nm, such as from about 360 nm to about 420 nm, such as from about 380 nm to about 400 nm or from about 400 nm to about 420 nm, such as about 365 nm or about 405 nm, for varying timespans. In some embodiments, the wavelength or wavelength range of UV light is about 350 nm to about 450 nm, such as from about 375 nm to about 425 nm. The wavelength or wavelength range can be constant or varying during operation 320. The source of the UV light can be the polymerization control device 105 described above. It is contemplated that other wavelengths of light can be used with appropriate reacting photoinitiators.

The polymerization conditions of operation 320 can further include a duration of exposure to the UV light. Such durations can be 1 millisecond (ms) or more and/or about 5 min. or less, such as from about 1 ms to about 60 seconds (s), such as from about 5 milliseconds to about 50 seconds, such as from about 50 milliseconds to about 45 seconds, such as from about 100 milliseconds to about 40 seconds, such as from about 0.5 seconds to about 30 seconds, such as from about 1 second to about 20 seconds. Shorter or longer durations of exposure to UV light are contemplated.

An energy density of the UV light for the polymerization conditions of operation 320 can be from about 1 mW/cm² to about 10,000 mW/cm², such as from about 10 mW/cm² to about 1,000 mW/cm², such as from about 50 mW/cm² to about 500 mW/cm², such as from about 75 mW/cm² to about 150 mW/cm², such as from about 80 mW/cm² to about 120 mW/cm². Higher or lower energy densities are contemplated. The energy density can be constant or varying during operation 320.

The polymerization process described herein can improve beta cell viability over conventional techniques. For example, upon photoinitiation, a homogenous hydrogel network with reduced network contraction relative to other equivalent materials reduces stress imparted on encapsulated/dispersed beta cells. In addition, it is believed that the polymerization described herein can mitigate ROS through active participation in the cross-linking mechanism of, e.g., PEGNB, contributing to the polymerization of the network rather than removing electrons from cellular membranes and destabilizing them, which is what kills or contributes to cell death. In polymerizations with PEGDA, ROS can be mitigated by purging oxygen from the microenvironment via a non-reactive or inert gas which is free or substantially free of oxygen can be used, such as nitrogen and noble gases (e.g., argon). For polymerizations using mixtures of PEGDA and PEGNB, ROS can be mitigated by the addition of PEGNB and its above properties, but can be further mitigated if necessary through purging of the microenvironment with inert gas.

In some cases, the combination of PEGNB with another photoreactive monomer, such as PEGDA, enables physical and chemical tuning of the droplet environment to optimize cell viability and excretion of, e.g., cytokines. The encapsulation/dispersion process and resultant hydrogel can maintain beta cell viability longer than non-encapsulated/dispersed counterparts, and can localize beta cells at a target location by temporarily preventing their migration.

After polymerization, the hydrogel-encapsulated/dispersed beta cells (which can be in the form of beta cells, beta-cell spheroids, and/or beta-cell spheroid like structures), and/or compositions comprising the hydrogel-encapsulated/dispersed beta cells (which can be in the form of beta cells, beta-cell spheroids, and/or beta-cell spheroid like structures), can be purified or otherwise isolated from the other materials exiting the microfluidic device.

In some embodiments, the plurality of beta cells, beta-cell spheroids, beta-cell spheroid-like structures, or combinations thereof dispersed in or encapsulated within a hydrogel have improved viability or lifetime relative to conventional methods. For example, the beta cells, beta-cell spheroids, beta-cell spheroid-like structures, or combinations thereof dispersed in or encapsulated within a hydrogel as described herein have cell viability of about 1 hour or more after encapsulation/dispersion, such as about 5 hours or more, such as about 10 hours or more, such as about 24 hours or more, such as about 36 hours or more, such as about 48 hours or more, such as about 60 hours or more, such as about 72 hours or more, such as about 84 hours or more, such as about 96 hours or more, such as about 108 hours or more, such as about 120 hours or more, such as about 132 hours or more, such as about 144 hours or more, such as about 156 hours or more, such as about 168 hours or more, such as about 180 hours or more, such as about 192 hours or more, 204 hours or more, such as about 216 hours or more, such as about 228 hours or more, such as about 240 hours or more after encapsulation/dispersion of the beta cells, beta-cell spheroids, beta-cell spheroid-like structures, or a combination thereof in the hydrogel. Shorter or longer time periods are contemplated.

The processes described herein can provide a high-throughput route to transplantable beta cells or beta-cell spheroids for the treatment of diabetes, and the resulting hydrogel-encapsulated/dispersed beta cells show, e.g., an enhanced ability to control insulin generation in response to glucose. Moreover, embodiments described herein enable aggregation of the beta cells, beta cell spheroids, beta-cell spheroid-like structures, or combinations thereof without the use of microwells.

In some embodiments, at least a portion of the plurality of beta cells dispersed in or encapsulated within a hydrogel form beta-cell spheroids, beta-cell spheroid-like structures, or a combination thereof. In these and other embodiments, the beta-cell spheroids, beta-cell spheroid-like structures, or a combination thereof can secrete insulin after encapsulation within and/or dispersion in the hydrogel. Secretion of insulin from the beta-cell spheroids, beta-cell spheroid-like structures, or a combination thereof can occur at about 1 hour or more after encapsulation/dispersion, such as about 5 hours or more, such as about 10 hours or more, such as about 24 hours or more, such as about 36 hours or more, such as about 48 hours or more, such as about 60 hours or more, such as about 72 hours or more, such as about 84 hours or more, such as about 96 hours or more, such as about 108 hours or more, such as about 120 hours or more, such as about 132 hours or more, such as about 144 hours or more, such as about 156 hours or more, such as about 168 hours or more, such as about 180 hours or more, such as about 192 hours or more, 204 hours or more, such as about 216 hours or more, such as about 228 hours or more, such as about 240 hours or more after encapsulation/dispersion of the beta-cell spheroids, beta-cell spheroid-like structures, or a combination thereof in the hydrogel. Shorter or longer time periods are contemplated.

Embodiments described herein can also enable control over hydrogel size and shape, via the manipulation of, e.g., relative flow velocity of immiscible phases, nozzle geometry, and nozzle dimension. The processes described herein promote long-term cell viability after encapsulation/dispersion, the dynamic adjustment and enhancement of glucose sensitivity and insulin secretion, as well as the protection of encapsulated/dispersed beta-cell spheroids from external deleterious factors such as innate immune responses and shear stress. The microparticle length scale also enables enhanced exchange of nutrients, waste, and secreted biomolecules to and from the beta cells and its surrounding environment, in contrast to other conventional encapsulation/dispersion methods.

The hydrogels which encapsulate/disperse the beta cells can have an average diameter of about 1 μm to about 2000 μm, such as from about 2 μm to about 1000 μm, such as from about 4 μm to about 500 μm, as determined by ImageJ (National Institutes of Health). In at least one embodiment, the hydrogels which encapsulate/disperse the beta cells can have an average diameter of about 500 μm or less, such as from about 50 μm to about 450 μm, such as from about 100 μm to about 400 μm, such as from about 150 μm to about 350 μm, such as from about 200 μm to about 300 μm. In some embodiments, the hydrogel can have an average diameter of about 50 μm to about 200 μm, such as from about 100 μm to about 180 μm or from about 75 μm to about 125 μm.

Adjusting the initial cell titer as well as channel dimensions, flowrates, photoreactive monomers, and linkers, as described herein can enable control of microparticle size (e.g., average diameter) and beta cell concentration in an independent manner. The aqueous phase in the following non-limiting embodiments refers to the phase of the hydrogel forming solution (which can include the beta cells).

(a) For hydrogels having an average diameter of about 250 μm and using a linker having a molecular weight of about 1000 to about 2000 Da, channel dimensions (h×w) for the oil phase can be from ˜75 μm×˜30 μm to ˜125 μm×˜50 μm, such as from ˜90 μm×˜35 μm to ˜110 μm×˜45 μm, such as ˜100 μm×˜40 μm; channel dimensions (h×w) for the aqueous phase can be from ˜75 μm×˜75 μm to ˜125 μm×˜125 μm, such as from ˜90 μm×˜90 μm to ˜110 μm×˜110 μm, such as ˜100 μm×˜100 μm; a flow rate of the oil phase can be from about 4 μL/min to about 8 μL/min, such as from about 5 μL/min to about 7 μL/min, such as from about 6 μL/min to about 6.5 μL/min; and/or a flow rate of the aqueous phase can be from about 4 μL/min to about 6 μL/min, such as from about 4.5 μL/min to about 5 μL/min or from about 5 μL/min to about 5.5 μL/min.

(b) For hydrogels having an average diameter of about 350 μm and using a linker having a molecular weight of about 1000 to about 2000 Da, channel dimensions (h×w) for the oil phase can be from ˜75 μm×˜30 μm to ˜125 μm×˜50 μm, such as from ˜90 μm×˜35 μm to ˜110 μm×˜45 μm, such as ˜100 μm×˜40 μm; channel dimensions (h×w) for the aqueous phase can be from ˜75 μm×˜75 μm to ˜125 μm×˜125 μm, such as from ˜90 μm×˜90 μm to ˜110 μm×˜110 μm, such as ˜100 μm×˜100 μm; a flow rate of the oil phase can be from about 2.8 μL/min to about 5.5 μL/min, such as from about 3.5 μL/min to about 5 μL/min, such as from about 3.8 μL/min to about 4.5 μL/min; and/or a flow rate of the aqueous phase can be from about 4 μL/min to about 6 μL/min, such as from about 4.5 μL/min to about 5 μL/min or from about 5 μL/min to about 5.5 μL/min.

(c) For hydrogels having an average diameter of about 450 μm and using a linker having a molecular weight of about 1000 to about 2000 Da, channel dimensions (h×w) for the oil phase can be from ˜125 μm×˜30 μm to ˜175 μm×˜50 μm, such as from ˜140 μm×˜35 μm to ˜160 μm×˜45 μm, such as ˜150 μm×˜40 μm; channel dimensions (h×w) for the aqueous phase can be from ˜125 μm×˜125 μm to ˜175 μm×˜175 μm, such as from ˜140 μm×˜140 μm to ˜160 μm×˜160 μm, such as ˜150 μm×˜150 μm; a flow rate of the oil phase can be from about 4 μL/min to about 8 μL/min, such as from about 5 μL/min to about 7 μL/min, such as from about 6 μL/min to about 6.5 μL/min; and/or a flow rate of the aqueous phase can be from about 2 μL/min to about 4 μL/min, such as from about 2.5 μL/min to about 3.5 μL/min.

(d) For hydrogels having an average diameter of about 250 μm and using a linker having a molecular weight of about 3000 to about 4000 Da, channel dimensions (h×w) for the oil phase can be from ˜75 μm×˜30 μm to ˜125 μm×˜50 μm, such as from ˜90 μm×˜35 μm to ˜110 μm×˜45 μm, such as ˜100 μm×˜40 μm; channel dimensions (h×w) for the aqueous phase can be from ˜75 μm×˜75 μm to ˜125 μm×˜125 μm, such as from ˜90 μm×˜90 μm to ˜110 μm×˜110 μm, such as ˜100 μm×˜100 μm; a flow rate of the oil phase can be from about 3.5 μL/min to about 5.5 μL/min, such as from about 4 μL/min to about 5 μL/min, such as from about 4 μL/min to about 4.5 μL/min; and/or a flow rate of the aqueous phase can be from about 4 μL/min to about 6 μL/min, such as from about 4.5 μL/min to about 5 μL/min or from about 5 μL/min to about 5.5 μL/min.

(e) For hydrogels having an average diameter of about 350 μm and using a linker having a molecular weight of about 3000 to about 4000 Da, channel dimensions (h×w) for the oil phase can be from ˜125 μm×˜30 μm to ˜175 μm×˜50 μm, such as from ˜140 μm×˜35 μm to ˜160 μm×˜45 μm, such as ˜150 μm×˜40 μm; channel dimensions (h×w) for the aqueous phase can be from ˜125 μm×˜125 μm to ˜175 μm×˜175 μm, such as from ˜140 μm×˜140 μm to ˜160 μm×˜160 μm, such as ˜150 μm×˜150 μm; a flow rate of the oil phase can be from about 4 μL/min to about 8 μL/min, such as from about 5 μL/min to about 7 μL/min, such as from about 6 μL/min to about 6.5 μL/min; and/or a flow rate of the aqueous phase can be from about 2 μL/min to about 4 μL/min, such as from about 2.5 μL/min to about 3.5 μL/min.

(f) For hydrogels having an average diameter of about 350 μm and using a linker having a molecular weight of about 3000 to about 4000 Da, channel dimensions (h×w) for the oil phase can be from ˜125 μm×˜30 μm to ˜175 μm×˜50 μm, such as from ˜140 μm×˜35 μm to ˜160 μm×˜45 μm, such as ˜150 μm×˜40 μm; channel dimensions (h×w) for the aqueous phase can be from ˜125 μm×˜125 μm to ˜175 μm×˜175 μm, such as from ˜140 μm×˜140 μm to ˜160 μm×˜160 μm, such as ˜150 μm×˜150 μm; a flow rate of the oil phase can be from about 3 μL/min to about 5 μL/min, such as from about 3.5 μL/min to about 4.5 μL/min; and/or a flow rate of the aqueous phase can be from about 4 μL/min to about 6 μL/min, such as from about 4.5 μL/min to about 5 μL/min or from about 5 μL/min to about 5.5 μL/min.

Beta cell concentrations within the hydrogel (e.g., suspended, dispersed, encapsulated, retained, or otherwise held in the hydrogels) can range from about 1 beta cell per hydrogel to thousands of beta cells per hydrogel, or more.

In some embodiments, a hydrogel encapsulates, disperses, suspends, retains, or otherwise holds from about 3 beta cells to about 100 beta cells, such as from about 10 beta cells to about 80 beta cells, such as from about 20 beta cells to about 70 beta cells, such as from about 30 beta cells to about 60 beta cells, such as from about 40 beta cells to about 50 beta cells.

The compositions formed by embodiments described herein can be in the form of a microcapsule. This microcapsule can include a core and a polymeric shell which at least partially encloses the core. The core includes a beta cell or a plurality of beta cells. The polymeric shell of the microcapsule is formed by the polymerization processes described herein.

In some embodiments, the compositions described herein include a first component and a second component. The first component can include a hydrogel and the second component can include a plurality of beta cells (e.g., two or more beta cells) encapsulated, dispersed, suspended, retained, or otherwise held in the first component.

In some embodiments, which can be combined with other embodiments, at least a portion of the beta cells of the hydrogel-encapsulated/dispersed beta cells are in the form of beta-cell spheroids, beta-cell spheroid-like structures, or a combination thereof.

As described above, isolated beta cells require contact with other beta cells to form beta-cell spheroids—or mimicry of such contact—to maintain viability and function. Recognizing this requirement, embodiments described herein can encourage or increase cell-cell contact for beta cells to form beta-cell spheroids. These beta-cell spheroids can mimic the function(s) of the body's natural glucose-controllers, e.g., the insulin-secreting beta cells of the pancreas. Here, processes described herein to form the hydrogel-encapsulated/dispersed beta cells can allow control over the beta-cell aggregation into well-defined cluster sizes. The bio-inertness of the hydrogel can provide a non-cytotoxic environment, and the hydrogel properties can be adjusted depending on application.

The processes for forming beta cells and/or beta cell spheroids dispersed/encapsulated in a hydrogel are a significant improvement over the existing state-of-the-art, as existing methods are lower in throughput or adversely affect beta-cell viability and function. The processes enable scaled beta cell spheroid production for producing spheroids at appropriate scale for transplantation procedures to treatment diabetes. The hydrogel can serve to modify beta cell behavior and/or optimize the therapeutic performance of beta cells by encapsulating or dispersing the beta cells. The cellular parameters, such as glucose sensitivity and insulin production, can be tuned via hydrogel-encapsulation and/or dispersion of the beta cells. Such processes and compositions are a significant improvement over the existing state-of-the-art, as existing methods have no way of predictively controlling such behavior.

Embodiments described herein also relate to uses of the compositions described herein such as for the treatment of a disease in a subject, as a platform for drug discovery or drug screening, among other applications.

In some embodiments, methods for treating a disease in a subject (e.g., an individual) includes administering to the subject one or more of the compositions described herein (e.g., the hydrogel-encapsulated/dispersed beta cells, beta-cell spheroids, and/or beta-cell spheroid-like structures). These compositions can secrete a therapeutically effective amount of a substance to treat a disease. For example, the compositions comprising the hydrogel-encapsulated/dispersed beta cells, beta-cell spheroids, beta-cell spheroid-like structures, or combinations thereof can secrete an amount (e.g., a therapeutically effective amount) of insulin to treat diabetes in the subject. In some embodiments, the encapsulated/dispersed beta cells, beta-cell spheroids, beta-cell spheroid-like structures, or combinations thereof can secrete insulin over a period over a period of about 1 day or more, such as about 2 days or more, such as about 3 days or more, such as about 5 days or more, such as about 7 days or more, such as about 10 days or more.

In some embodiments, a method of providing beta cells to an individual in need thereof can include administering to the individual an effective amount of a composition described herein. Individuals in need of beta cells can include individuals having diabetes.

The hydrogel droplets can enable the beta cells to be injected in a minimally invasive manner (e.g., through a syringe) analogous to “naked” beta cells. This can remove the need for surgical procedures and can greatly reduce the chance of complications and patient recovery time. Also the droplets can maintain superior oxygenation of encapsulated/dispersed beta cells and can enable superior waste removal from the immediate cell environment, as opposed to a “bulk” hydrogel containing beta cells. This can be due to the superior surface area to volume ratio which facilitates rapid diffusion between the encapsulated beta cell and the surrounding environment.

As described above, beta cells are a type of cell found in pancreatic islets. Because of the short lifespan of pancreatic islets outside of the body, conventional methods for diabetes research, drug discovery, and drug screening using such islets can be challenging. For example, because islets and beta cells vary in size and cellular composition, multiple islets and beta cells are typically pooled for each and every experimental condition tested. Such pooling can involve hand-picking of the individual islets and beta cells resulting in high costs. Further, the insulin secretory function of the beta cells can be influenced by the individual islets and/or individual beta cells selected such that there is high batch-to-batch variation. Such challenges restrict high-throughput drug screening and disease modeling. The compositions described herein and processes for forming such compositions can be utilized to solve these and other issues because, e.g., the compositions and syntheses thereof enable large-scale production of functional and viable beta cells.

In some embodiments, a method for screening a pharmaceutical or a material utilized in the diagnosis or treatment of disease such as diabetes can include a substance that increases or decreases insulin secretion to one or more compositions described herein, and monitoring the amount of insulin secretion. Substances include materials that increase or decrease insulin secretion such as glucose, an incretin, acetylcholine, agonists, antagonists, inhibitors, derivatives thereof, mimetics thereof, or combinations thereof. Illustrative, but non-limiting, examples of substances can additionally, or alternatively, include norepinephrine, somatostatin, galanin, prostaglandins, derivatives thereof, mimetics thereof, or combinations thereof. The amount of insulin secretion can be monitored by various techniques including ELISA, real-time polymerase chain reaction (RT PCR), mass spectroscopy, raman spectroscopy, spectrophotometry, or combinations thereof.

The following examples are put forth so as to provide those of ordinary skill in the art with a complete disclosure and description of how to make and use embodiments of the present disclosure, and are not intended to limit the scope of embodiments of the present disclosure. Efforts have been made to ensure accuracy with respect to numbers used (e.g. amounts, dimensions, etc.) but some experimental errors and deviations should be accounted for.

### EXAMPLES

The assembly of beta cells into spheroid or spheroid-like structures was examined. The hydrogel-encapsulated/dispersed beta cells of the present disclosure were compared to the conventional technique of utilizing microwell cell cultures (comparative example) to form beta cell spheroids. In the Examples section, the comparative example and comparative method is the in-vitro assembled, beta-cell spheroids formed using microwells. The examples illustrate the superiority of embodiments described herein relative to microwell technology to, e.g., promote assembly of the beta cells into beta-cell spheroid and/or beta-cell spheroid-like structures, and enhance the survival of the beta cells. In the FIGS., 1.5 k linker refers to the 1500 Da PEG-dithiol linker, and 3.5 k linker refers to the 3500 Da PEG-dithiol linker

### Cell Culture

Beta-TC-6 (MIN6) cells were purchased from American Type Culture Collection (ATCC, CRL-11506, USA). Cells were cultured at a temperature of about 37° C. under an atmosphere of about 5% CO₂ in a culture media containing Dulbecco's Modified Eagle's Medium (DMEM, Life Technologies, USA) with a high-glucose supplement, about 15 vol % heat-inactivated fetal bovine serum (FBS), and about 1 vol % antibiotic-antimycotic (Life Technologies, USA). The culture media was changed every ˜3 days and cell populations were sub-cultured every ˜6 days. To prepare the beta cells for encapsulation/dispersion in the hydrogels, the beta cells were detached from culture flasks with TrypLE™ Express (Life Technologies, USA), pelleted, and re-suspended to a desired concentration, e.g., about 10³ to about 10⁷, in the culture media.

### Test Methods

1. Equilibrium Hydrogel Property Measurements. To quantify the hydrogel swelling ratio, about 30 μL hydrogel forming solution composed of about 10 wt % PEGNB, 10 mM of either 1500 Da PEG-thiol linker (polyethylene glycol dithiol, Sigma Aldrich, USA) or 3500 Da PEG-dithiol linker (polyethylene glycol dithiol Jenkem, China), and about 0.1 wt % LAP was photopolymerized within a 1 mL syringe with the tip cut off for about 20 seconds under about 100 mW/cm². Then hydrogel samples were incubated at about 37° C. in Dulbecco's Phosphate Buffered Saline (DPBS, pH ˜7.4), then collected at fixed intervals (e.g., daily for ˜5 days), and their mass after swelling was measured. The dry mass of the hydrogel samples was measured after lyophilization for about 24 hours. The swelling ratio was determined as the ratio between hydrogel dry and swelling mass. Flory-Rehner calculations were used for determining hydrogel mesh size. To measure hydrogel elastic modulus, mechanical testing was performed under monotonic compression using a Dynamic Mechanical Analyzer (DMA Q800, TA, DE, USA). Here, the samples were placed between two compression platens and a ˜0.003 N preload was applied to ensure contact between the sample and the moving platen. The sample length was measured from the distance between the fixed platen and the moving platen. The compressive load was applied in a linear ramp fashion at a loading rate of ˜0.05 N/min for the samples with the 1500 Da PEG-dithiol linker and a loading rate of ˜0.1 N/min for the samples with the 3500 Da PEG-dithiol linker.

2. Cell Viability Assays. The viability of the encapsulated/dispersed beta cells and beta-cell spheroids was measured by staining using a live/dead viability kit (Life Technologies, USA), which is a cellular membrane integrity assay that stains live cells with green fluorescence and dead cells with red fluorescence. Cell viability was imaged with an inverted fluorescence microscope (IX-71, Olympus, USA) and cell viability was measured as the ratio of number of live cells to total cells using ImageJ. About 100 single beta cells and 50 beta-cell spheroids were imaged for cell viability, which was shown as mean cell viability±standard deviation.

3. Immunofluroescent Staining. The in-vitro assembled beta-cell spheroid structures using microwells (comparative examples) and the example beta-cell-laden hydrogel microspheres were stained to confirm the presence of intracellular insulin and E-cadherin for evidence of cell-cell interactions. In-vitro assembly of beta-cell spheroids in microwells was conducted to determine the minimum number of cells to form a spheroid. Beta-cell spheroid assembly in microwells was compared to the assembly of beta cells encapsulated/dispersed in the hydrogel microspheres to investigate any differences in the behavior of cells between the two processes.

The cells or hydrogel microspheres/dispersions were fixed about 5 days after seeding into microwells or micro-encapsulation/dispersion in about 4 wt % paraformaldehyde for ˜15 minutes at about room temperature, rinsed with Phosphate Buffered Saline (PBS) for ˜5 minutes, then blocked with about 5 wt % bovine serum albumin (BSA) for ˜1 hour at about room temperature to prevent nonspecific binding. After washing 3 times with PBS, the cells or hydrogel microspheres were incubated in a guinea pig polyclonal anti-insulin primary antibody (˜1:25 part dilution, ThermoFisher Scientific, USA), and a mouse monoclonal primary antibody against E-cadherin (˜1:150 part dilution, BD Transduction, USA) at about 4° C. for a time period of about 8-24 hours. After rinsing 3 times with 5% BSA, the cells or hydrogel microspheres were incubated in corresponding secondary antibodies (Alexa Fluor™ 488 donkey-antimouse IgG and Alexa Fluor™ 633 goat-anti-guinea pig IgG, ˜10 μg/mL, ThermoFisher Scientific, USA) at about 4° C. for a time period of about 8-24 hours. The cells or hydrogel microspheres were then rinsed 3 times again with about 5 wt % BSA, and then incubated in about 1 μg/mL 4′-6-diamidino-2-phenylindole (Sigma Aldrich, USA) to counterstain the nuclei. The cells or hydrogel microspheres were imaged using a spinning disc super resolution confocal microscope with fluorescent lasers (SpinSR10, Olympus, USA).

4. Insulin Secretion. A static glucose stimulated insulin release (GSIR) assay was performed to quantify insulin secretion. Secreted insulin was measured on day 1 and day 5 post-encapsulation/dispersion via an enzyme-linked immunosorbent assay (ELISA) per a suitable protocol such as a manufacturer's protocol. Briefly, the beta-cell-laden hydrogel microspheres were preconditioned in a Krebs-Ringer Buffer (KRB) containing about 2 mM glucose for a time period of about 1 hour. The beta-cell-laden hydrogel microspheres were then transferred into a KRB containing about 25 mM glucose and were maintained in the buffer for a period of about 1 hour. Secreted insulin was then measured using a mouse insulin sandwich ELISA kit (Sigma, USA). The same batch of beta-cell-laden microgels was then lysed and double-stranded deoxyribonucleic acid (dsDNA) was extracted per a suitable protocol such as the manufacturer's protocol (Invitrogen, USA). Briefly, a working solution was made by diluting Quant-iT™ dsDNA BR reagent 1:200 in Quant-iT™ dsDNA BR buffer. For example, for ˜100 assays, ˜100 μL of Quant-iT™ dsDNA BR reagent (Component A) and ˜20 mL of Quant-iT™ dsDNA BR buffer (Component B) were placed in a disposable plastic container and mixed. About 200 μL of the working solution was loaded into each microplate well. ˜10 μL of each DNA standard was added to separate wells and mixed. ˜1-20 μL of each unknown DNA sample was added to separate wells and mixed. The fluorescence was measured using a microplate reader, and a standard curve was used to determine the amounts of DNA. The secreted insulin was normalized to the dsDNA content.

5. Statistical Analysis. Unpaired student's t-tests and one-way analysis of variance (ANOVA) (GraphPad Prism) were used to determine statistical significance (*p<0.05) in analyzing the theoretical mesh size over time between hydrogels made with either the 1500 Da PEG-dithiol linker or the 3500 Da PEG-dithiol linker, and insulin secretion from beta-cell-laden microgels made from either the 1500 Da PEG-dithiol linker or the 3500 Da PEG-dithiol linker. Results were presented as mean±standard deviation of three samples.

### A. Comparative Example

### 1. Fabrication of Agarose Microwells and In-Vitro Assembly of Beta-Cell Spheroids.

Microwells with flat bottoms have been fabricated to assemble beta-cell spheroids. However, flat-bottomed wells are constrained by their inability to assemble beta-cell spheroids from very few beta cells, and therefore factors governing spheroid assembly remain largely unexplored. In addition, although substrate hydrophobicity-induced self-assembly of beta-cell spheroids has been demonstrated, but self-assembly techniques lack control over the size of the formed beta-cell spheroids. Moreover, cellular interactions within beta-cell spheroids may be altered in response to changes in hydrophobicity, which does not recapitulate in vivo growth conditions. Microwells with concave bottoms were used to address these concerns and still allow finite control over beta-cell spheroid size.

2 wt % agarose (Sigma Aldrich, USA) was dissolved in 0.9 w/v % NaCl on a heat block. Then, 500 μL of dissolved agarose was transferred into 3D Petri Dishes (Microtissues, USA). The gelled agarose microwell pad was peeled off from the mold after cooling down, and soaked into Dulbecco's Modified Eagle's Medium (DMEM, Life Technologies, USA) with 15 vol % fetal bovine serum (FBS, Life Technologies, USA), and 1 vol % antibiotics (e.g., 10,000 units/mL of Penicillin, 10,000 μg/mL of Streptomycin and 25 μg/ml amphotericin B in saline solution) for a time period of 8-24 hours before use.

The beta cells were then seeded into concave-bottom 296-well agarose molds. Here, 190 μL of beta-cell containing culture media with a desired cell density was added into the microwells. Dilutions containing 2×10⁴ cells/mL, 3×10⁴ cells/mL, 5×10⁴ cells/mL, 6×10⁴ cells/mL, and 8×10⁴ cells/mL were used for seeding. The seeded microwells were then transferred into 6-well plates supplemented with culture media and antibiotics, then cultured in an incubator at 37° C. under an atmosphere of 5% CO₂ (0.75 atm to 0.8 atm). Beta-cell assembly was monitored every day via microscopy (IX-71 fluorescent microscope Olympus, USA). The number of seeded cells per well on day 1 and an average diameter of formed beta-cell spheroids on day 5 was measured using ImageJ (National Institutes of Health). After culturing for 5 days, the seeded cells were stained for viability measurements.

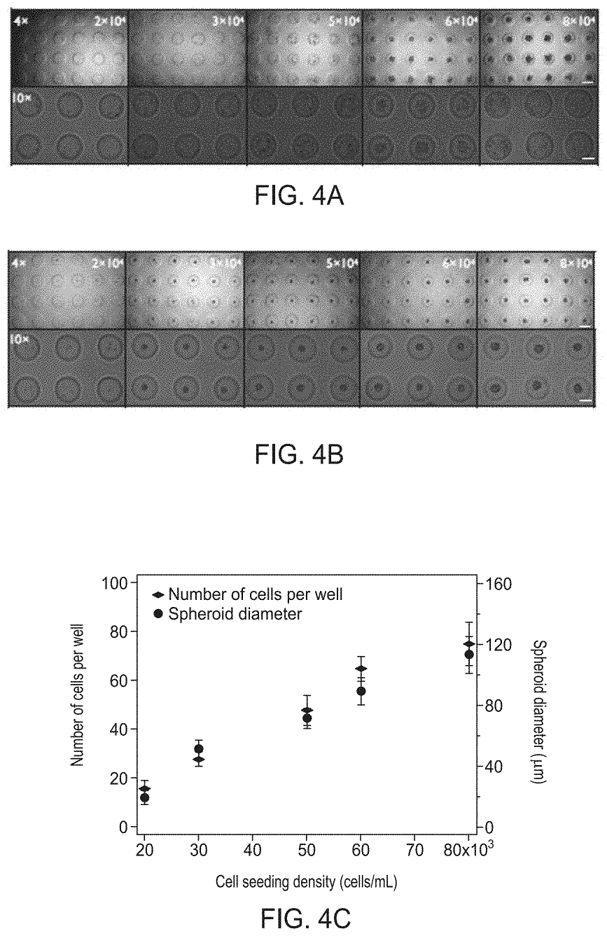

FIGS. 4A-4C show data and images for the in-vitro assembly of example beta-cell spheroids made by the comparative method. The images in FIG. 4A and FIG. 4B show beta-cell seeding on day 1 and on day 5, respectively, at 2×10⁴, 3×10⁴, 5×10⁴, 6×10⁴, and 8×10⁴ cells/mL from left to right. The images on the top panel of both FIGS. 4A and 4B were captured with a 4× objective lens, scale bar 400 μm, and the images on the bottom panel of both FIGS. 4A and 4B were captured with a 10× objective lens, scale bar 200 μm. FIG. 4A shows that the average number of cells seeded per well increased with increasing cell-seeding density. As shown in FIG. 4B, after 5 days of culture, the beta cells sedimented onto the bottom of each well and aggregated to form spheroid structures with rounded shape and a diameter that positively correlated with the average number of cells seeded per well on day 1. FIG. 4C shows results from varying the cell-seeding density. The average number of beta cells received per well on day 1 that formed a spheroid structure on day 5 was measured with ImageJ. Seeding densities of 2×10⁴, 3×10⁴, 5×10⁴, 6×10⁴, and 8×10⁴ beta cells/mL resulted in beta-cell spheroid structures with an average diameter of 20 μm, 50 μm, 70 μm, 89 μm, and 113 μm, respectively, 5 days after incubation.

### 2. Beta-Cell Viability in Microwells as a Function of Cell Seeding Density

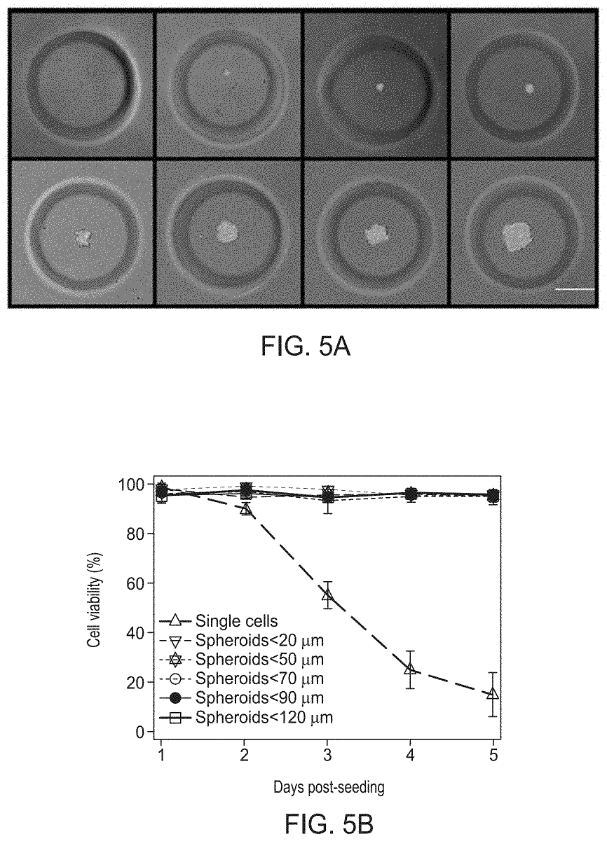

To determine the minimum number of beta cells capable of forming a beta-cell spheroid or spheroid-like structure, and whether the size of the cellular structure affects long-term beta-cell viability, beta-cell-seeding density was gradually diminished until each well received as few as one beta cell or two beta cells, to as many as tens of beta cells. FIGS. 5A and 5B show results with respect to the survival of the in-vitro assembled beta-cell spheroids/spheroid-like structures made by the comparative method. Specifically, the fluorescent images of FIG. 5A (scale bar 200 μm) illustrate the viability of beta cells or beta-cell spheroid/spheroid-like structures on day 5 with increasing diameter. The live cells stain green and the dead cells stain red.

After about 5 days of culture, most single beta cells were dead. This poor beta-cell viability was rescued when three beta cells were able to make contact and form a beta-cell spheroid/spheroid-like structure. Additionally, even with concave-bottom microwells, the frequency of observing beta-cell spheroid/spheroid-like structure assemblies with only two beta cells was low. However, the frequency to observe small beta-cell spheroid/spheroid-like structures with a diameter below 30 μm was good, and once a beta-cell spheroid/spheroid-like structure was formed, cell viability proved to be independent of the size of the beta-cell spheroid structure (FIG. 5B). These results indicate that enhanced cellular tolerance and functionality was established through cell-cell interactions.

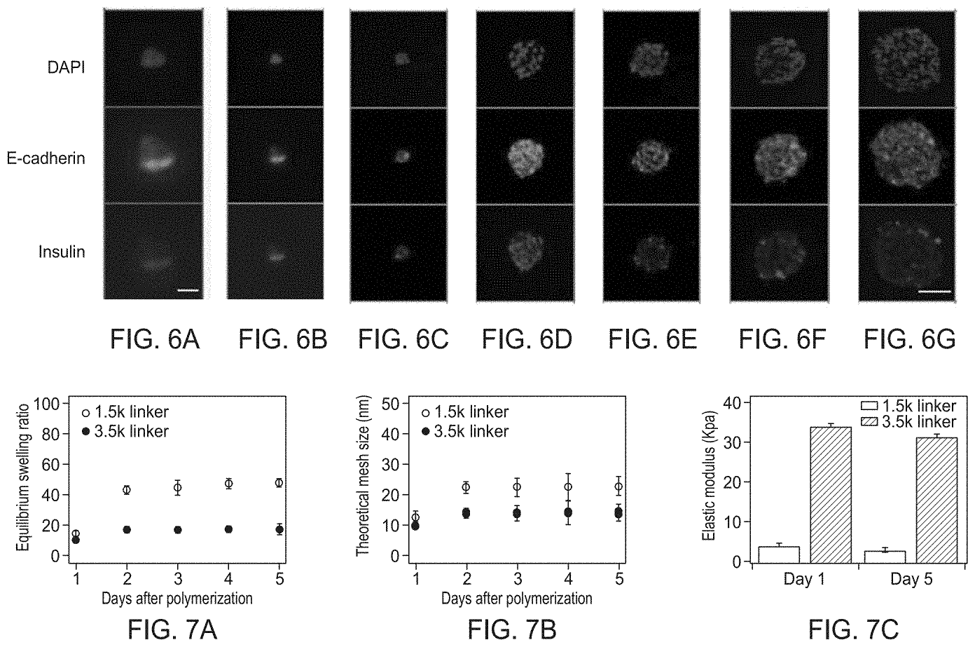

This result was validated by the presence of E-cadherin and intracellular insulin as shown by the images (FIGS. 6A-6G) of the in-vitro assembled beta-cell spheroid/spheroid-like structures made by the comparative method. In FIGS. 6A-6G, DAPI refers to 4′,6-diamino-2-phenylindole.

Specifically, FIG. 6A (40× objective lens; scale bar: 10 μm) and FIG. 6B (20× objective lens) shows the spheroid/spheroid-like structure formed by three beta cells. FIGS. 6B-6G (scale bar: 50 μm) show images of immunofluorescent staining of various beta-cell spheroid/spheroid-like structures of a range of sizes. The images and results can explain a beta-cell spheroid assembly principle where as long as cell-cell contact is permitted, regardless of the number of beta cells, beta-cell spheroid/spheroid-like structures can be formed and can have long-term cell viability. Direct cell-cell contact did not guarantee beta-cell spheroid/spheroid-like structure formation. Since beta-cell spheroid formation depends heavily on cell-cell interactions via cell-adhesive molecules on the extracellular membrane, this result may indicate that the interaction between these cell-adhesive molecules is a factor in maintaining beta-cell viability, and can be strong enough to recruit several single beta cells together into a cluster. This technique could also be used to inform the assembly conditions for cellular spheroids formed by other cell types, including, e.g., hepatic stellate cells, breast cancer cells, and stem cells.

### B. Example Hydrogel-Encapsulated/Dispersed Beta Cells

### 1. Example Macromer and Photoinitiator Synthesis

The hydrogel forming macromer, PEGNB, was synthesized according to established procedures. Briefly, about 10 g of 4-arm PEG (MW, 20,000 Da, JenKem Technology, China) was placed in a 250 mL round-bottom flask containing about 20 mL of MeCl₂ and dissolved at about room temperature (15° C.-25° C.) while stirring. Into a separate 50 mL round-bottom flask was added about 0.54 g of N,N-dicyclohexylcarbodiimide (DCC, Sigma Aldrich, USA) followed by dissolution in about 10 mL of MeCl₂. To this solution was added dropwise about 0.70 g of 5-norbornen-2-carboxylic acid (5N2B, Sigma Aldrich, USA). The contents were placed in an argon environment and stirred for about 30 minutes at about room temperature whereupon the initially clear solution turned cloudy white. To the 250 mL round-bottom flask containing fully dissolved PEGNB was added about 0.21 g of pyridine and about 0.032 g of 4-dimethylamino pyridine (Sigma Aldrich, USA). The 250 mL round-bottom flask was then fitted with a medium-mesh fritted glass filter (Ace glass) and a vacuum adapter and set in an ice bath. The contents of the 50 mL round-bottom flask were filtered via vacuum into the 250 mL round-bottom flask, stirred, and allowed to react under argon for about 24 hours. The product-containing solution was washed twice with 5% NaHCO₃, then precipitated in ice-cold diethyl ether. The precipitate was filtered on a Buchner funnel, and then placed in a Soxhlet extractor fitted with an Allihn condenser and washed with gently-boiling ether for about 48 hours. The product was removed from the extractor and lyophilized for about 24 hours. The degree of functionalization (greater than about 90%) was confirmed via 400 MHz proton nuclear magnetic resonance (NMR) using d2-DMSO as solvent.

The initiator species, lithium acylphosphinate (LAP), was synthesized according to the following procedure. About 3.0 g of 2,4,6-trimethylbenzoyl chloride (Sigma Aldrich, USA) was added dropwise to a 250 mL round-bottom flask containing an equimolar amount of dimethyl phenylphosphonite and stirred at about room temperature under nitrogen for about 8 to 24 hours. An about four-fold excess of lithium bromide (LiBr, Sigma Aldrich, USA) dissolved in about 100 mL of methyl ethyl ketone (MEK, Sigma Aldrich, USA) was added to the round-bottom flask and the resulting mixture was heated to about 50° C. for about 10 minutes. White crystalline salts were formed upon cooling to about room temperature over a period of about 8 to 24 hours. Product crystals were filtered on a Buchner funnel and rinsed with ice-cold MEK, then placed under vacuum until a constant weight was achieved. LAP was confirmed via 400 MHz proton nuclear magnetic resonance (NMR) using a suitable deuterated solvent such as deuterated toluene (toluene-d8).

### 2. Example Fabrication of the Microfluidic Device

Microfluidic flow networks were fabricated using standard soft lithography techniques. Briefly, about 20 g polydimethylsiloxane (PDMS) was poured onto a silicon wafer (Silicon Inc., USA) that had been photolithographically-patterned with microscale flow channels, vacuumed for about 30 minutes to remove the entrapped air, and then transferred to an oven (temperature of about 70° C.) to cure for about 8 to 24 hours. PDMS replicas were then trimmed and punched with a sharpened 20 G dispensing needle (CML Supply, USA) to fashion inlets and outlets. After sonication in ethanol, the PDMS replicas were exposed to oxygen plasma (Harrick Scientific, USA), placed in conformal contact with clean glass slides, and transferred to an oven (temperature of about 70° C.), and remained in the oven for about 8 to 24 hours to form an irreversible bond between the PDMS microfluidic replica and the glass slide or glass coverslip. Two channel dimensions (height×width, h×w) were specifically fabricated to vary the microgel size: (1) 100 μm×40 μm for the oil phase, and 100 μm×100 μm for the aqueous phase (e.g., the hydrogel forming solution phase); and (2) 150 μm×40 μm for the oil phase, and 150 μm×150 μm for the aqueous phase (e.g., the hydrogel forming solution phase).

### 3. Example Beta-Cell Microencapsulation/Dispersion

In two example formulations, the hydrogel forming solution containing ˜10 wt % 20,000 Da 4-arm PEGNB, ˜10 mM 1500 Da PEG-dithiol linker or 3500 Da PEG-dithiol linker, and ˜0.1 wt % LAP was mixed gently together with suspended beta cells before injection into a microfluidic device, where the beta-cell-containing hydrogel forming solution was hydrodynamically pinched by a fluorocarbon oil to generate droplet emulsions. Upon ultraviolet (UV) light exposure at about 100 mW/cm² for about 20 seconds or about 5 seconds for the 1500 Da PEG-dithiol linker or 3500 Da PEG-dithiol linker, respectively, droplet emulsions were photopolymerized into microgels. The wavelength of the UV light was about 290 nm to about 500 nm. Due to the viscosity difference caused by the variation in molecular weight, the hydrogel forming solution composed of the 3500 Da PEG-dithiol linker typically had a higher viscosity than the 1500 Da PEG-dithiol linker. Thus, a slight variation in flow rates was applied when the two linkers were used to form microgels of specific size.

The following example, non-limiting, parameters can be utilized to adjust the size of the microgels (e.g., the hydrogel-encapsulated/dispersed beta cells). Here, the aqueous phase refers to the phase of the hydrogel forming solution.

(1) To make microgels having an average diameter of ˜250 μm using the 1500 Da PEG-dithiol linker, channel dimensions (h×w) were about 100 μm×about 40 μm for the oil phase, and about 100 μm×about 100 μm for the aqueous phase. Flow rates for the oil phase and aqueous phase were held constant at about 6.5 μL/min and about 5 μL/min, respectively.

(2) To make microgels with an average diameter of ˜350 μm using the 1500 Da PEG-dithiol linker, channel dimensions (h×w) were about 100 μm×about 40 μm for the oil phase, and about 100 μm×about 100 μm for the aqueous phase. Flow rates for the oil phase and aqueous phase were held constant at about 3.6 μL/min and about 5 μL/min, respectively.

(3) To make microgels with an average diameter of ˜450 μm using the 1500 Da PEG-dithiol linker, channel dimensions (h×w) were about 150 μm×about 40 μm for the oil phase, and about 150 μm×about 150 μm for the aqueous phase. Flow rates for the oil phase and the aqueous phase were held constant at about 6 μL/min and about 3 μL/min, respectively.

(4) To make microgels with an average diameter of ˜250 μm using the 3500 Da PEG-dithiol linker, channel dimensions (h×w) were about 100 μm×about 40 μm for the oil phase, and about 100 μm×about 100 μm for the aqueous phase. The flow rates for the oil phase and the aqueous phase were held constant at about 4.5 μL/min and about 5 μL/min, respectively.

(5) To make microgels having an average diameter of ˜350 μm using the 3500 Da PEG-dithiol linker, channel dimensions (h×w) were about 150 μm×about 40 μm for the oil phase, and about 150 μm×about 150 μm for the aqueous phase. The flow rates for the oil phase and the aqueous phase were held constant at about 6 μL/min and about 3 μL/min, respectively.

(6) To make microgels having an average diameter of ˜450 μm using the 3500 Da PEG-dithiol linker, channel dimensions (h×w) were about 150 μm×about 40 μm for the oil phase, and about 150 μm×about 150 μm for the aqueous phase. The flow rates for the oil phase and the aqueous phase were held constant at about 4 μL/min and about 5 μL/min, respectively.

(7) To encapsulate/disperse beta-cell spheroids, channel dimensions (h×w) were about 100 μm×about 100 μm for the beta-cell spheroid-containing hydrogel forming solution, and about 100 μm×about 40 μm for the oil carrier phase. The flow rates for the oil phase and the aqueous phase were held constant at about 4 μL/min and about 5 μL/min, respectively.

To encapsulate/disperse in-vitro assembled beta-cell spheroids, the formed beta-cell spheroids were recovered and suspended in the hydrogel forming solution about 5 days after cell seeding. This was performed to, e.g., observe whether in-vitro assembled beta-cell spheroids assembled via microwells could also be encapsulated with high viability.

The hydrogel microspheres were recovered into the aqueous phase by centrifugation on a 40 μm cell strainer (ThermoFisher Scientific, USA), and cultured in culture media for monitoring long-term cell viability.

### C. Results

1. Differential Mechanical Property of the Hydrogels Made with the 1500 Da PEG-Dithiol Linker and the 3500 Da PEG-Dithiol Linker

The length of the dithiol linkers can result in, e.g., various gelation efficiencies in thiol-ene reactions and hydrogel property changes over time. To quantitatively determine the differences in hydrogel property, the elastic modulus, swelling ratio, and estimated mesh size were measured and calculated over a period of about 5 days. Such properties are shown by the exemplary data of FIGS. 7A-7C. Theoretically, having a longer linear molecular backbone should result in hydrogels having a larger mesh size and a lower mechanical strength. On the contrary, and as shown in FIG. 7B, hydrogels made from the 1500 Da PEG-dithiol linker showed a significant increase in mesh size than those with the 3500 Da PEG-dithiol linker due to a lower gelation efficiency. The hydrogels made from the 1500 Da PEG-dithiol linker also had higher softness and swelling ratio than those with the 3500 Da PEG-dithiol linker as shown in FIGS. 7A and 7C. After incubation for about 5 days, the swelling ratio, mesh size, and elastic modulus were further increased in hydrogels made with the 1500 Da PEG-dithiol linker. Hydrogels made with the 3500 Da PEG-dithiol linker, however, showed minimal changes in the same parameters over about 5 days.

These results indicated that, under identical reaction conditions, the singular change of linker length can significantly affect hydrogel formation rates and hydrogel mechanical properties. These differences can result from, e.g., progression of hydrogel network architecture as they are crosslinked by dithiol linkers of different length. Shorter linkers can be more likely to react with -enes from the same PEGNB molecule, presumably due to their inability to reach a neighboring PEGNB molecule, leading to “linker-neutralization” and little or no crosslinking. Additionally, disulfide formation and “self-termination” of the linker may be present at higher rates for the 1500 Da PEG-dithiol linker than for the 3500 Da PEG-dithiol linker. Such linker self-termination can reduce crosslinking and can produce a stoichiometric mismatch between thiols and -enes in the hydrogel-forming solution. The result of this can be the larger hydrogel mesh size formed using the 1500 Da PEG-dithiol linker than the 3500 Da PEG-dithiol linker. This unintuitive result is used to control cellular fate and function in novel ways as described herein. For example, tuning of the hydrogel properties is utilized to enable, e.g., long-term beta-cell viability, beta-cell spheroid assembly, and optimization of both glucose sensitivity and insulin secretion—all key parameters to the successful and effective treatment of diabetes.

### 2. Microencapsulation/Dispersion of Beta Cells and Beta-Cell Spheroids

To understand factors that may affect cytocompatibility in hydrogel microspheres, droplet size and cell loading number per droplet were decoupled and analyzed separately. By manipulating the nozzle dimension of the droplet generator and the relative flow rates of the immiscible phases, microgels with various diameters were fabricated—about 250 μm, about 350 μm, and about 450 μm.

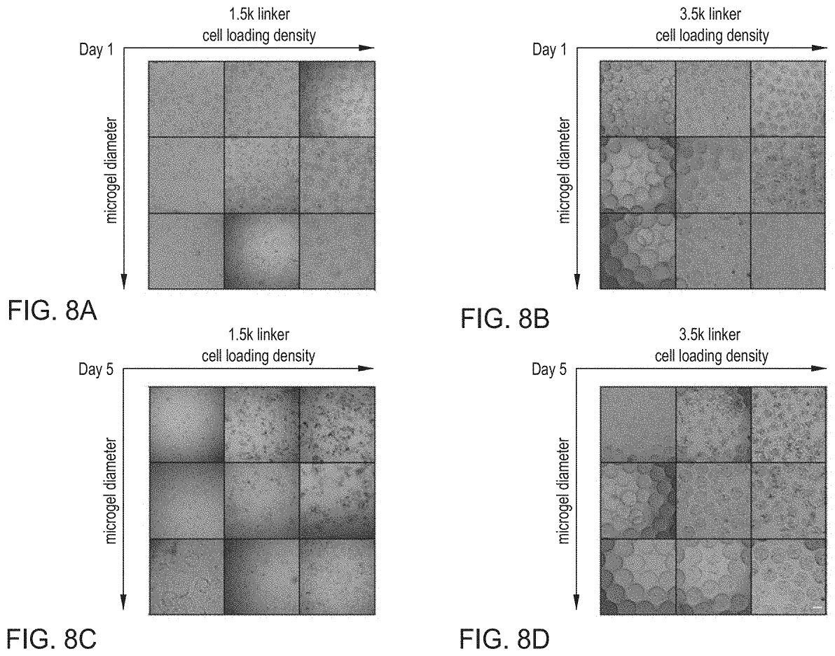

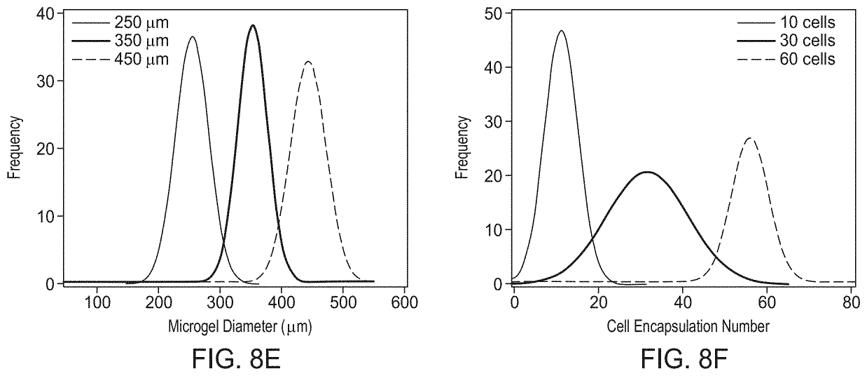

FIGS. 8A-8D and FIGS. 8E-8G show exemplary images and data, respectively for the microencapsulation/dispersion of beta cells within example PEGNB microgels. As described above, the beta cells can be in the form of beta cells, beta-cell spheroids, and/or beta-cell spheroid-like structures. Specifically, the images of FIGS. 8A-8D (scale bar: 200 μm) show the encapsulated/dispersed beta cells within microgels on day 1 and day 5. In each section of images, the beta-cell-loading density increases from left to right, and the microgel diameter increases from top to bottom. Accordingly, the number of beta cells encapsulated/dispersed per microgel was controlled by varying the beta-cell-loading density. As shown by the exemplary data of FIG. 8E and FIG. 8F, the microgel diameter and the number of beta cells per microgel can be well-controlled by embodiments described herein. Here, a higher precision can be achieved when encapsulating/dispersing fewer than ˜15 beta cells within a microgel than when encapsulating/dispersing ˜30 beta cells or ˜60 beta cells, which may be a result from cell aggregates formation when the beta-cell-loading density is too high (FIG. 8G).

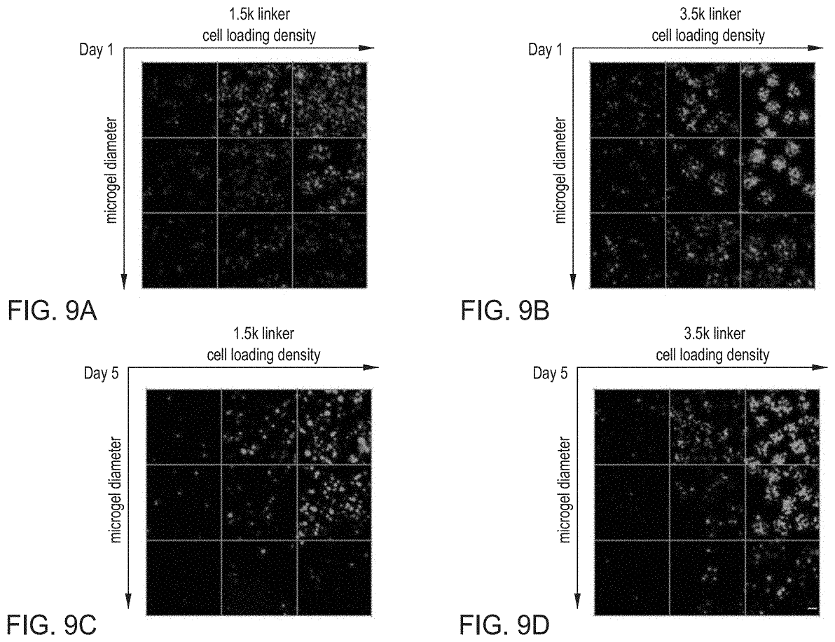

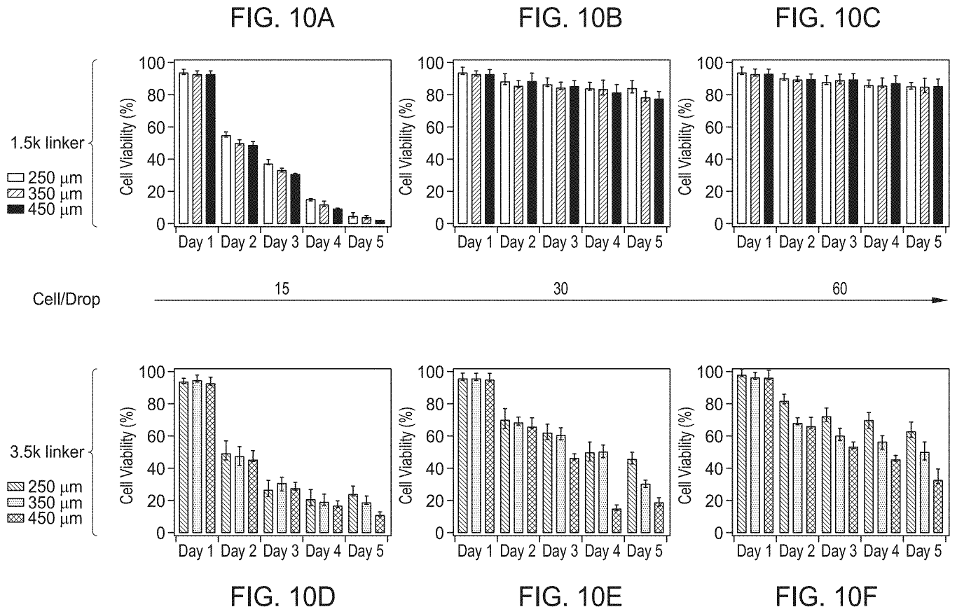

FIGS. 9A-9D and FIGS. 10A-10F show results with respect to beta-cell viability after encapsulation/dispersion for the example microgel-encapsulated/dispersed beta cells (e.g., the hydrogel-encapsulated/dispersed beta cells). Specifically, the images in FIGS. 9A-9D (scale bar: 200 μm) show the initial and long-term beta-cell viability as a function of beta-cell-loading density and microgel diameter. Here, the live cells stain green and the dead cells stain red. In each section of the images of FIGS. 9A-9D, the beta-cell-loading density increases from left to right, and the microgel diameter increases from top to bottom.

FIGS. 10A-10F show exemplary data of the quantified beta-cell viability over time for the microgels of varying diameters made from the 1,500 Da (1.5 k) linker and the 3,500 Da (3.5 k) linker. In FIGS. 10A-10F, “cell/drop” refers to the number of cells per droplet or number of cells per microgel. For example, 15, 30, and 60 refer to 15 cells per microgel, 30 cells per microgel, and 60 cells per microgel, respectively.

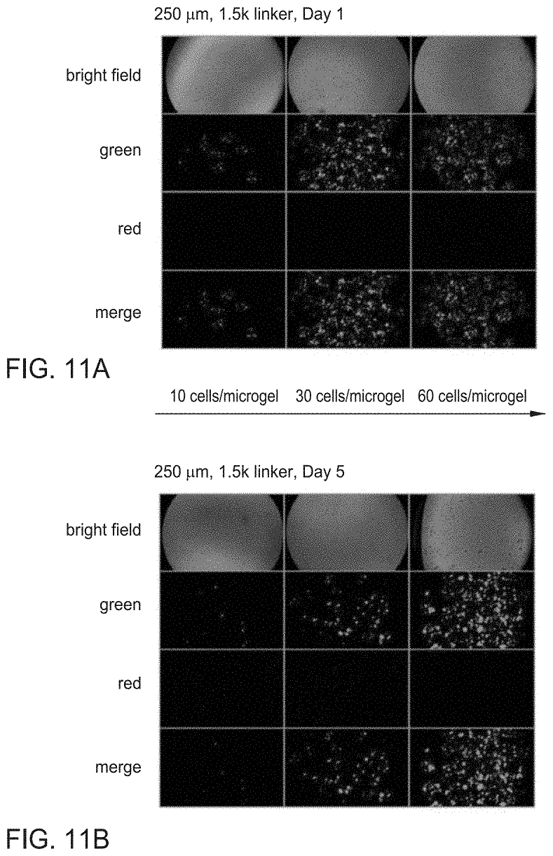

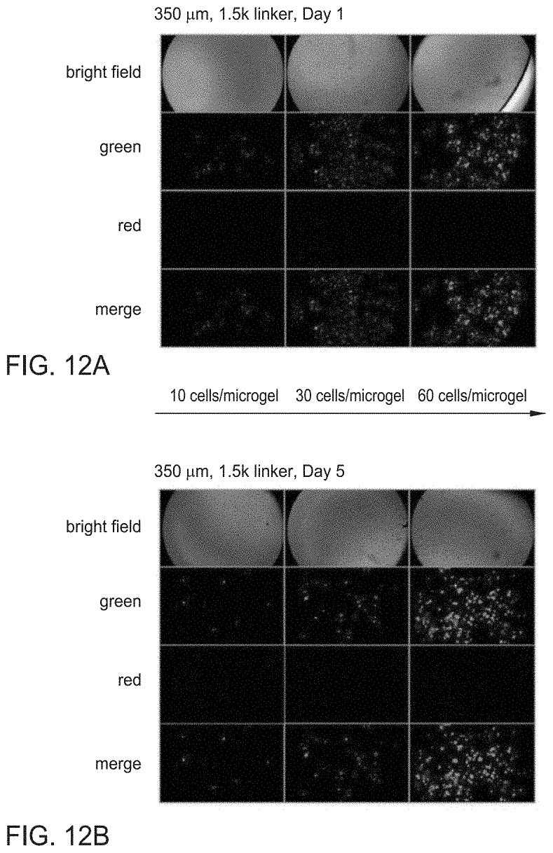

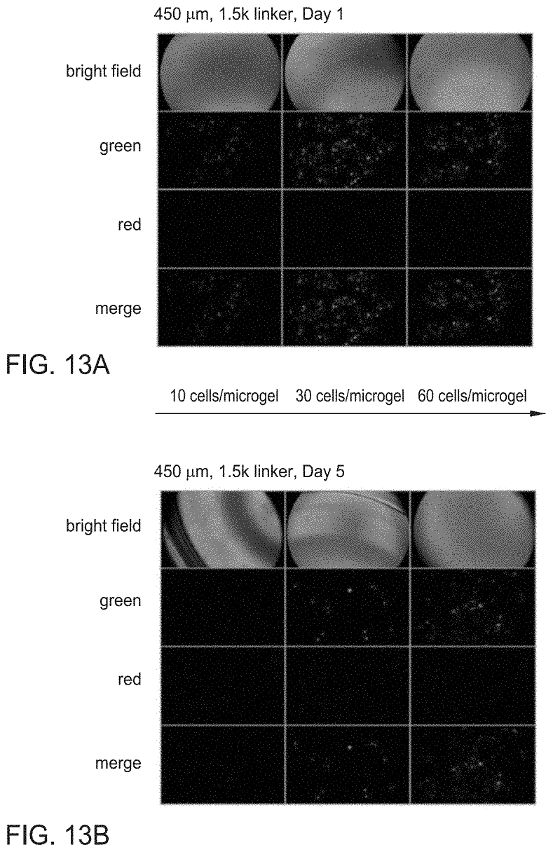

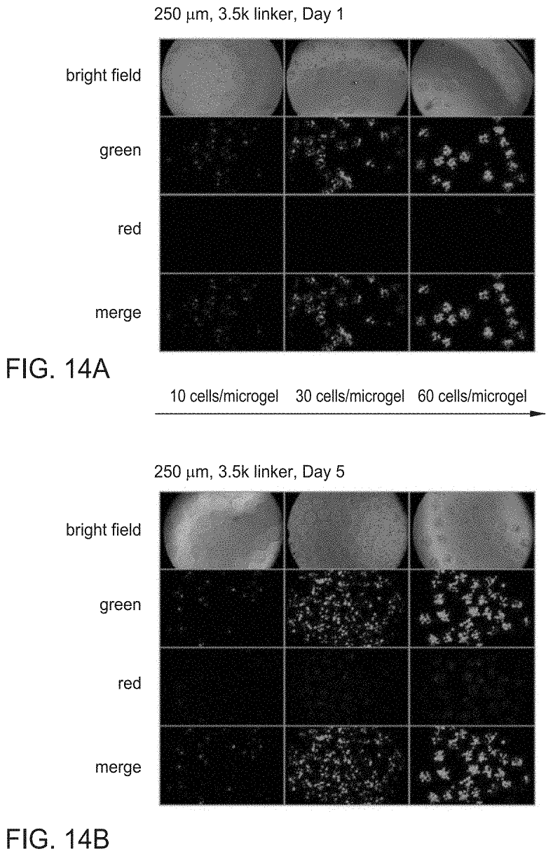

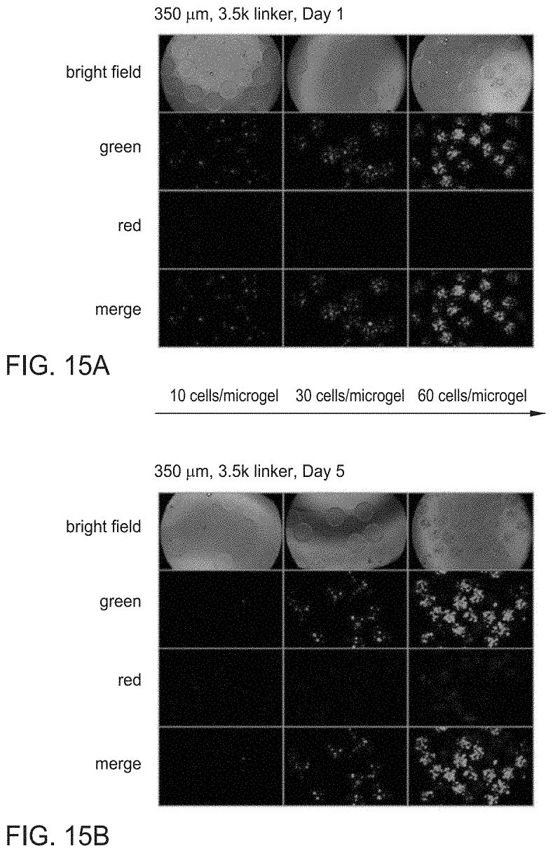

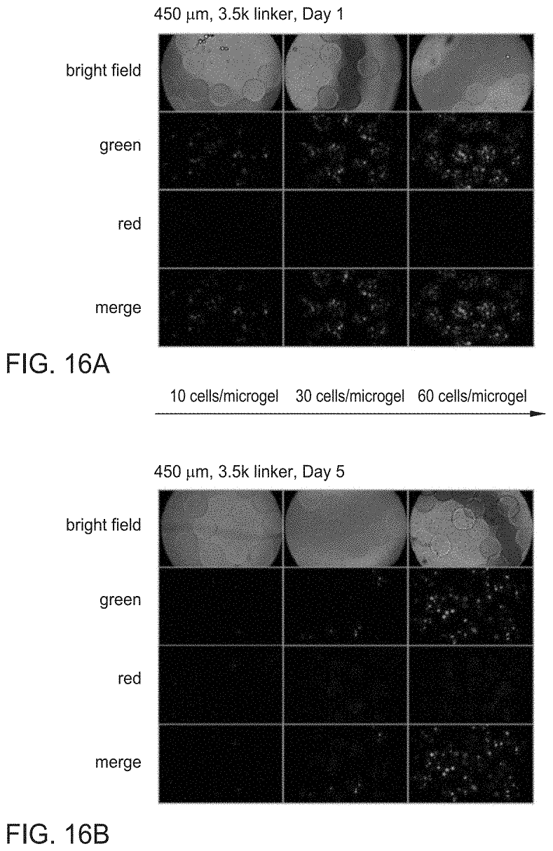

With the 1500 Da PEG-dithiol linker, when the beta-cell-loading number per droplet was fixed while increasing the droplet size, the beta-cell viability decreased over time, even though the initial beta-cell viability can be maintained at a very high level. When the droplet size was fixed while increasing the beta-cell-loading number per droplet, the beta-cell viability and the frequency of beta-cell spheroid formation increased (FIGS. 9A-9D and FIGS. 10A-10F). These results are further supported by the bright field and fluorescent images of FIGS. 11A-16B, showing beta-cell viability on day 1 and day 5 within microgels of varying properties such as average diameter (˜250 μm, ˜350 μm, and ˜450 μm), PEG-dithiol linker (1500 Da and 3500 Da), and the number of beta cells per microgel (˜10, ˜30, and ˜60).

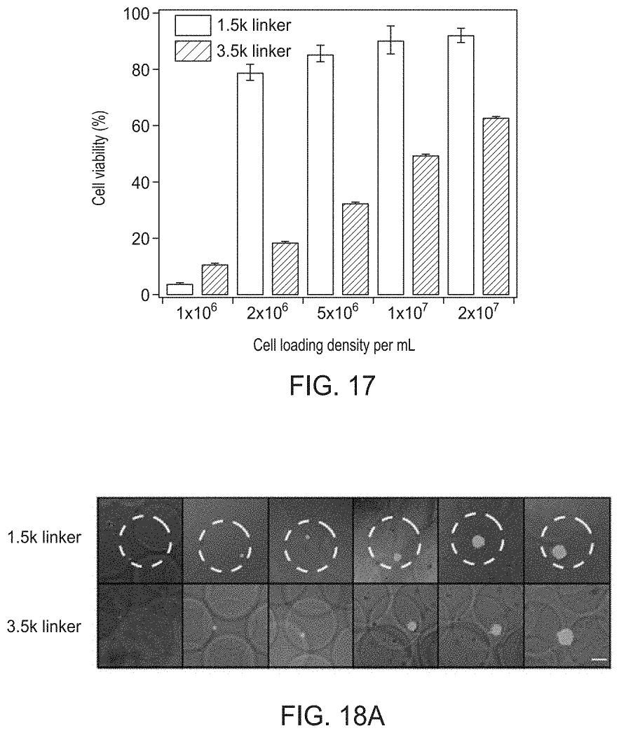

FIG. 17 shows exemplary data for beta-cell viability as a function of beta-cell-loading density for the example microgels. The data was measured for microgels made from the 1,500 Da (1.5 k) linker or the 3,500 Da (3.5 k) linker, having a beta-cell-loading density of ˜1×10⁶ beta cells/mL, ˜2×10⁶ beta cells/mL, ˜5×10⁶ beta cells/mL, ˜1×10′ beta cells/mL, or ˜2×107⁶ beta cells/mL. The results show a linear correlation between the beta-cell viability and the beta-cell-loading density, where the beta-cell viability on day 5 increases as the beta-cell-loading density increases. With the 3500 Da PEG-dithiol linker, the beta-cell-loading density positively affected cell viability on day 5, but not as much as that observed with the 1500 Da PEG-dithiol linker.

These results indicate beta-cell viability can be affected by the beta-cell-loading density, possibly via paracrine signaling, which is limited with microgels having a low beta-cell-loading density. Cell-cell contact in excess of 2 beta cells induces a beta-cell spheroid/spheroid-like structure assembly, which was heavily presented throughout the hydrogel microspheres/microgels made with the 1500 Da PEG-dithiol linker and the 3500 Da PEG-dithiol linker. The ability for beta cells to form groups of 2 or more, or 3 or more, and assume a spheroid or spheroidal-like conformation/structure, also significantly improves beta-cell viability.

Consistent with the microwell study, the hydrogel-encapsulated/dispersed single beta cells did not survive after 5 days of culture; instead, only living beta cells on day 5 were in the form of spheroids or spheroid-like structures. This result further indicates that the assembly of beta cells into beta-cell spheroid/spheroid-like structures can play a more important role than paracrine signaling in regulating cell-cell communications. In addition, studies have indicated that smaller droplet sizes having larger surface-to-volume ratios are more deleterious to cells in radical-initiated photopolymerizations due to the rapid diffusion of reactive oxygen species (ROS). The results presented herein show that the variation in droplet sizes did not affect initial cell viability, indicating that the polymerization of PEGNB can mitigate deleterious ROS.

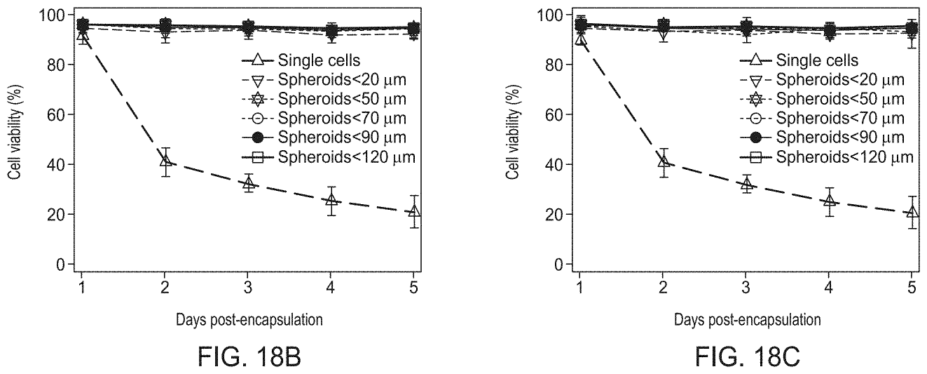

To further elucidate the long-term beta-cell viability within microgels as a function of beta-cell spheroid/spheroid-like structure assembly efficiency, in-vitro assembled beta-cell spheroid/spheroid-like structures of various sizes were encapsulated/dispersed in microgels made from the 1500 Da PEG-dithiol linker or the 3500 Da PEG-dithiol linker. FIGS. 18A-18C show exemplary images of, and exemplary data for, example microencapsulated/dispersed single beta cells and example in-vitro assembled beta-cell spheroid/spheroid-like structures. Specifically, FIG. 18A (scale bar: 100 μm) is a series of fluorescent images showing example microgels made from a 1500 Da PEG-dithiol linker or a 3500 Da PEG-dithiol linker encapsulating/dispersing single beta cells (first image from each row) or encapsulating/dispersing beta-cell spheroid/spheroid-like structures of a range of sizes on day 5. The live cells stain green and the dead cells stain red. The dashed circles designate the periphery of the microgels made from the 1500 Da PEG-dithiol linker due to the low contrast resulting from microgel swelling. FIG. 18B and FIG. 18C provide results regarding the beta-cell viability on day 5 with example microgels made from the 1500 Da PEG-dithiol linker and the 3500 Da PEG-dithiol linker, respectively. The results illustrate that, independent of the changes in its beta-cell spheroid/spheroid-like structure size and hydrogel properties, the encapsulated/dispersed beta-cell spheroid/spheroid-like structures showed about 100% beta-cell viability after 5 days. This indicates that the assembly of beta cells into beta-cell spheroids/spheroid-like structures is a factor in maintaining long-term beta-cell viability.

### 3. Expression of Intracellular Insulin and E-cadherin

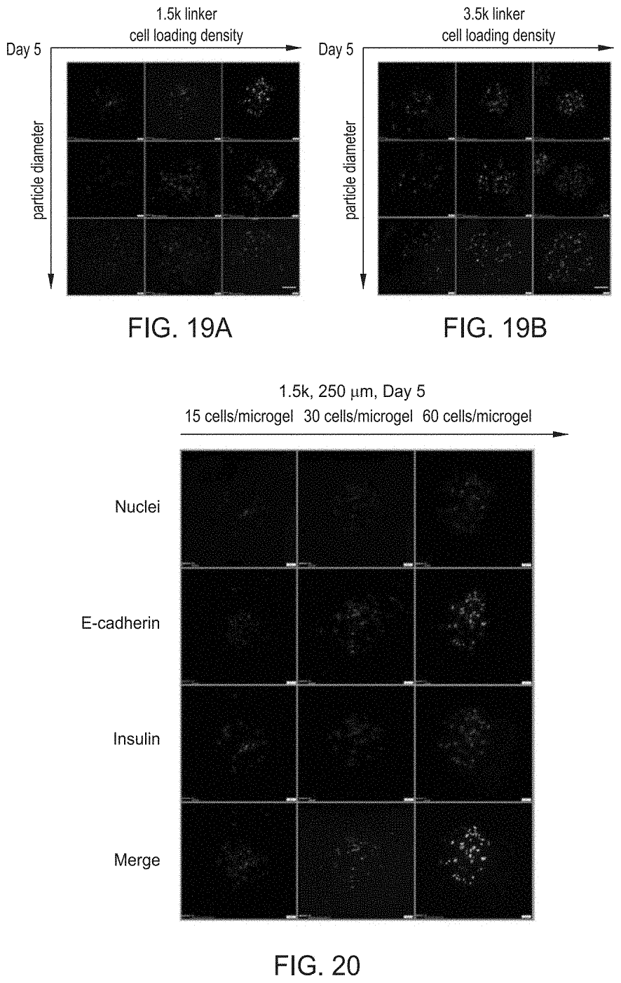

To further validate that the hydrogels/microgels made from the 1500 Da PEG-dithiol linker can promote beta-cell viability by direct cell-cell interactions, E-cadherin and intracellular insulin staining was performed on hydrogel-encapsulated/dispersed beta cells. FIG. 19A and FIG. 19B show exemplary immunostaining images of beta cells within example microgels made from the 1500 Da PEG-dithiol linker or the 3500 Da PEG-dithiol linker on culture day 5, respectively. The blue color represents nuclei, the green color represents E-cadherin, and the red color represents insulin. For the nine images shown in FIG. 19A and the nine images shown in FIG. 19B, the beta-cell-loading density increases from left to right, and the microgel diameter increases from top to bottom.

The results of FIGS. 19A and 19B indicate that as long as beta-cell spheroid/spheroid-like structures were formed within the hydrogel particles (e.g., microgels), the expression of E-cadherin and intracellular insulin could be observed regardless of the beta-cell loading density or the microgel diameter. Notably, when the beta-cell-loading density was high (e.g., about 2×10⁷ cells/mL), cells in microgels made with the 3500 Da PEG-dithiol linker also had highly expressed E-cadherin, but concentrated only where it supported regional cell-cell interactions. There were still a percentage of beta cells having no E-cadherin and very limited intracellular insulin expression. This may be due to limited cellular mobility in hydrogels made with the 3500 Da PEG-dithiol linker. Groups of 3 or more beta cells already close to each other were able to from beta-cell spheroids/spheroid-like structures for this expression to take place and the beta cells were inhibited from moving any appreciable distance. In addition, the beta cells in microgels made with the 1500 Da PEG-dithiol linker had very evenly distributed expression of E-cadherin throughout the network, and small spheroid/spheroid-like structures were formed locally and showed E-cadherin expression.

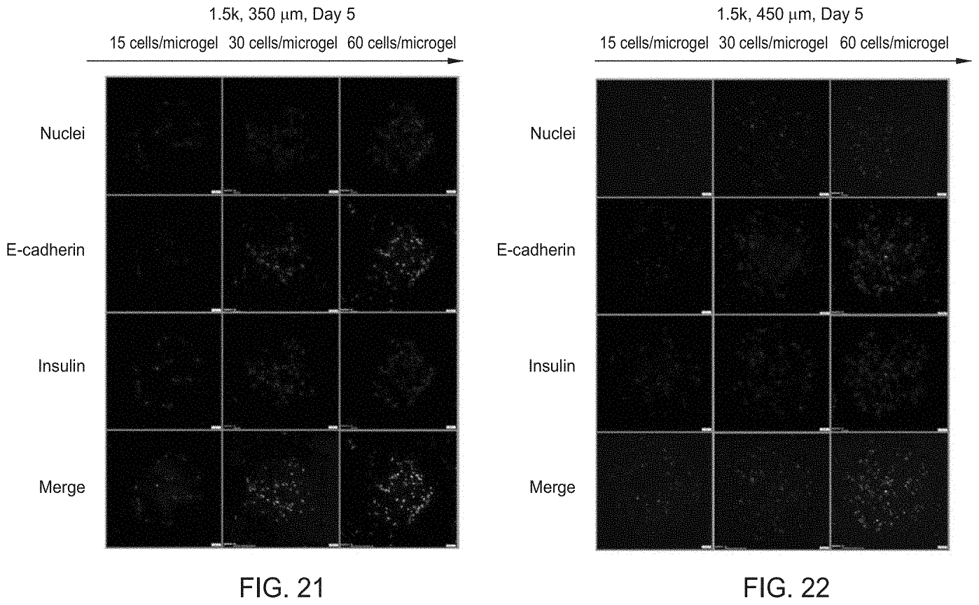

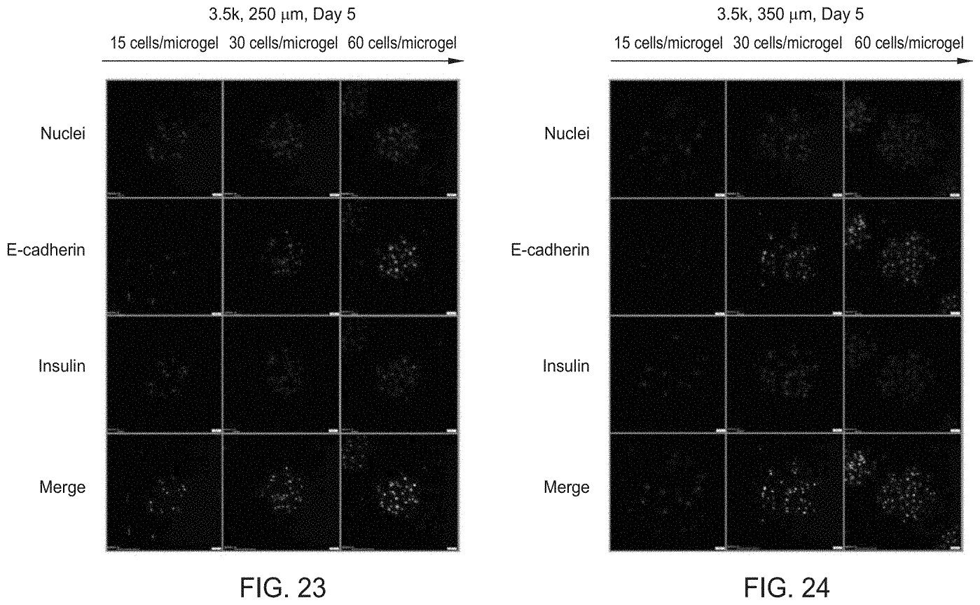

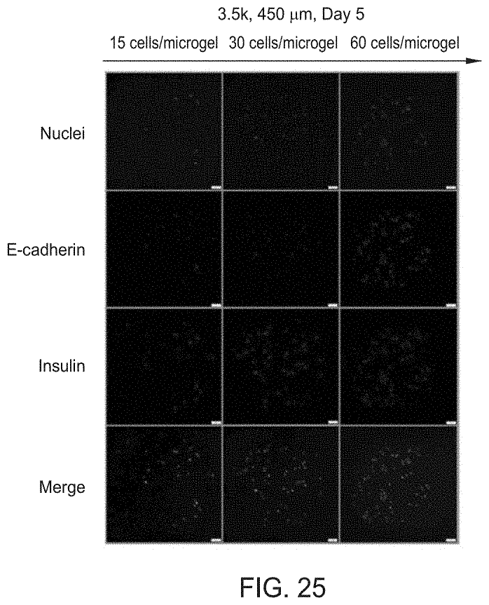

Each of FIGS. 20-25 include a series of exemplary images of individual and merged color channels showing nuclei (blue), E-cadherin (green), and intracellular insulin (red) expression of beta cells encapsulated/dispersed within example microgels. The exemplary images were captured on culture day 5. The microgels imaged had varying diameters (250 μm, 350 μm, or 450 μm) and were made of either a 1500 Da PEG-dithiol linker or a 3500 PEG-dithiol linker. The images of FIGS. 20-25 provide evidence that intracellular insulin was normally expressed for all microgels tested, as the beta-cell spheroid/spheroid-like structures showed a yellowish color when the channels were merged (FIGS. 20-25). Although all of the microgels showed formation of the beta-cell spheroid/spheroid-like structures, the larger mesh size of the microgels formed from the 1500 Da PEG-dithiol linker (relative to those formed from the 3500 Da PEG-dithiol linker) can result in a looser network architecture, enabling beta cells to easily migrate together to form beta-cell spheroids/spheroid-like structures. Because cell-cell interactions are a factor for beta-cell viability and functionality, the results can help explain the elevated beta-cell viability in microgels made with the 1500 Da PEG-dithiol linker.

### 4. Glucose Sensitivity of Encapsulated/Dispersed Beta Cells

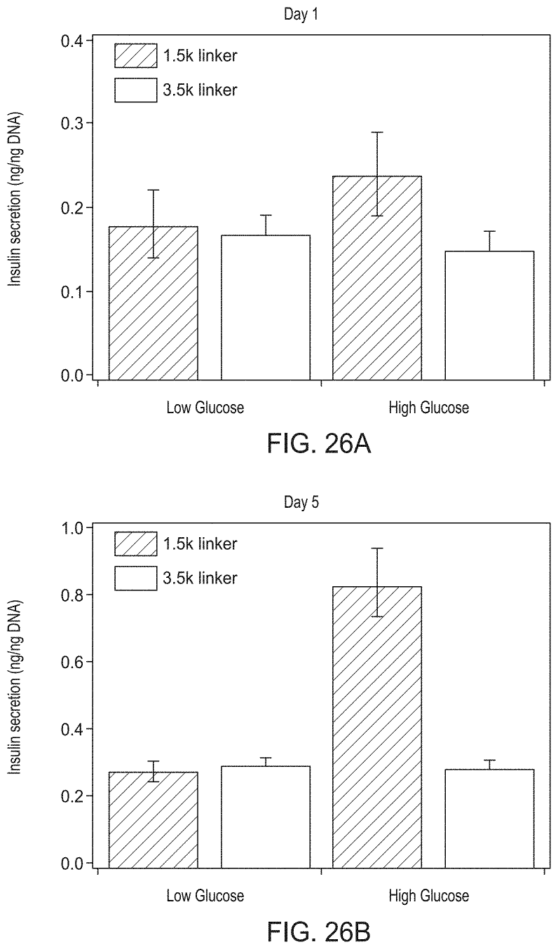

To investigate, e.g., whether enhanced cell-cell interactions and long-term beta-cell viability have a positive impact on the functionality of the encapsulated/dispersed beta cells, glucose-stimulated insulin secretion was quantitatively measured via ELISA. FIGS. 26A and 26B show bar graphs of insulin secretion from example beta-cell-laden microgels in response to glucose stimulation on day 1 (FIG. 26A) and day 5 (FIG. 26B). The microgels used for the experiment had a diameter of about 250 μm and were made from a 1500 Da PEG-dithiol linker or a 3500 Da PEG-dithiol linker.

The data presented in FIGS. 26A and 26B show that insulin secretion increased over time (comparing day 1 to day 5). Specifically, immediately after encapsulation or dispersion (day 1), the beta cells encapsulated/dispersed within microgels made with either the 1500 Da PEG-dithiol linker or 3500 Da PEG-dithiol linker showed moderate to good insulin secretion in response to glucose flux, and increasing glucose concentration did not alter this result significantly. However, after about 5 days of incubation and as shown in FIG. 26B, beta cells encapsulated/dispersed within microgels made with the 1500 Da PEG-dithiol linker had a higher insulin secretion index than beta cells encapsulated/dispersed within microgels made with the 3500 Da PEG-dithiol linker. The results indicate that locally auto-assembled beta-cell spheroids/spheroid-like structures can have improved long-term beta-cell viability and enhanced glucose-stimulated insulin secretion. Moreover, the beta cells secreted more insulin in response to high glucose levels, indicating an improved sensitivity to glucose flux.

Overall, the results can show that beta cells exhibit different responses to changes in hydrogel properties, and the beta-cell spheroid/spheroid-like structure assembly plays a role in regulating long-term viability and functionality of beta cells. As described herein, the use of droplet microfluidics to induce beta-cell spheroid/spheroid-like assembly within hydrogel enables the high-throughput fabrication of beta-cell spheroids/spheroid-like structures needed for clinical trials and patients. These cell-laden microgels are capable of secreting insulin continuously, and responding to glucose stimulation. Further, embodiments described herein are applicable to ‘artificial pancreas’ applications with continuous insulin sensing and regulatory secretion, thereby advancing current therapies and informing cell-based therapies for type 1 diabetes.

As described above, most clinically available therapies for type 1 diabetes are inadequate in monitoring and regulating glucose levels dynamically and conveniently. Macroscopic hydrogels (or macrogels) have been studied due to the feasibility to fabricate such hydrogels. However, surgical implantation of macrogels is challenging. In addition, the diffusional length scale in macrogels constrains their performance in vitro and ex vivo, by limiting bidirectional transport of nutrients, gases, and biological molecules from the center of the hydrogels.

In contrast, embodiments described herein enable the control of, e.g., hydrogel physical characteristics and the assembly of beta-cell spheroid/spheroid-like structures locally within microscopic hydrogels with comparable or superior beta-cell viability as those formed within traditional, macroscopic hydrogels or spheroid/spheroid-like structures assembled within microwells. In particular, the higher long-term beta-cell viability found with embodiments described herein could potentially reduce injection frequencies, thus making the therapy more cost-effective. Further, and as described herein, by using droplet-microfluidics, fabrication throughput can be significantly improved to satisfy the high volumes needed for clinical testing, which often require the use of large quantities of viable and functional beta cells.

Embodiments described herein enable assembly of beta cell-spheroids and/or spheroid like structures. In some examples, the combination of beta-cell assembly characteristics with droplet-microfluidics and degradable materials enables beta-cell spheroid structures to be assembled within hydrogel microspheres in a high-throughput fashion, with improved long-term cell viability, and glucose dependent insulin secretion. As a result, this high-throughput beta-cell spheroid assembly platform can be used for the creation of an ‘artificial pancreas’ where millions of such cell-laden hydrogel microspheres are employed as functional units within a complex, tunable continuous matrix. This technique can provide an alternative route to achieve insulin independence and normoglycemia for the treatment of type 1 diabetes.

The processes described herein enable the creation of cell-laden microparticles that maintain high viability—analogous to that of unencapsulated control—regardless of microparticle size. The processes also enable the encapsulated/dispersed beta cells to maintain this high level of viability on a long-term basis. The microparticle environment offers, e.g., a cross-linked hydrogel mesh network that can mimics the characteristics of a cell's natural endogenous extracellular matrix and cell-microenvironment effects.

The hydrogels or compositions comprising hydrogels described herein have a biocompatible microenvironment suitable for encapsulation and/or dispersion of living beta cells in sufficient quantities and are formed in rapid enough timespans to enable their therapeutic application in living organisms. The length scale of these hydrogel microenvironments makes them superior to other conventional technologies, enables optimal exchange of nutrients, waste, and secreted biomolecules to and from the cell and its surrounding environment, and enables their minimally invasive delivery via syringe injection.

In the foregoing, reference is made to embodiments of the disclosure. However, it should be understood that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the foregoing embodiments, features, aspects, and advantages are merely illustrative and are not considered elements or limitations of the appended claims except where explicitly recited in a claim(s). Likewise, reference to “the disclosure” shall not be construed as a generalization of any inventive subject matter disclosed herein and shall not be considered to be an element or limitation of the appended claims except where explicitly recited in a claim(s).

For purposes of this present disclosure, and unless otherwise specified, all numerical values within the detailed description and the claims herein are modified by “about” or “approximately” the indicated value, and consider experimental error and variations that would be expected by a person having ordinary skill in the art. For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

As used herein, the indefinite article “a” or “an” shall mean “at least one” unless specified to the contrary or the context clearly indicates otherwise. For example, embodiments comprising “a monomer” include embodiments comprising one, two, or more monomers, unless specified to the contrary or the context clearly indicates only one monomer is included.

While the foregoing is directed to embodiments of the present disclosure, other and further embodiments of the disclosure may be devised without departing from the basic scope thereof, and the scope thereof is determined by the claims that follow.

What is claimed is:

1. A composition, comprising:
a first component comprising a hydrogel, the hydrogel comprising, in polymerized form, one or more photoreactive monomers and a thiol linker; and
a second component comprising a plurality of beta cells dispersed or encapsulated within the hydrogel.

2. The composition of claim 1, wherein at least a portion of the plurality of beta cells are in the form of beta-cell spheroids, beta-cell spheroid-like structures, or a combination thereof.

3. The composition of claim 1, wherein:
the hydrogel is in the form of a microparticle; and
an average diameter of the microparticle is about 2,000 μm or less.

4. The composition of claim 1, wherein the one or more photoreactive monomers comprise a methylene functional group, an acid functional group, or combinations thereof.

5. The composition of claim 4, wherein, when the one or more photoreactive monomers comprise the methylene functional group, the one or more photoreactive monomers comprise polyethylene glycol norbornene, polyethylene glycol diacrylate, derivatives thereof, or combinations thereof.

6. The composition of claim 4, wherein, when the one or more photoreactive monomers comprise the acid functional group, the one or more photoreactive monomers comprise polylactic acid, derivatives thereof, or combinations thereof.

7. The composition of claim 1, wherein the thiol linker is a dithiol linker.

8. The composition of claim 7, wherein the dithiol linker is a polyethylene glycol-dithiol.

9. The composition of claim 1, wherein:
the thiol linker is a polyethylene glycol-dithiol linker having a molecular weight from about 500 Da to about 15,000 Da;
the one or more photoreactive monomers has a number average molecular weight from about 250 Da to about 50,000 Da; or
a combination thereof.

10. The composition of claim 1, wherein the one or more photoreactive monomers comprise polyethylene glycol norbornene having a molecular conformation of 1 arm to 12 arms.

11. A process for forming a composition, comprising:
introducing a plurality of beta cells with one or more components to form a reaction mixture, the one or more components comprising a photoreactive monomer, a photoinitiator, a dithiol linker, or combinations thereof;
introducing a fluorocarbon oil to the reaction mixture; and
polymerizing the reaction mixture by exposure to ultraviolet light, under polymerization conditions, to form the composition, the composition comprising the plurality of beta cells dispersed in or encapsulated within a hydrogel.

12. The process of claim 11, wherein:
at least a portion of the plurality of beta cells dispersed in or encapsulated within the hydrogel form beta-cell spheroids, beta-cell spheroid-like structures, or a combination thereof; and
the beta-cell spheroids, beta-cell spheroid-like structures, or a combination thereof secrete insulin after 24 hours.

13. The process of claim 11, wherein the photoreactive monomer comprises polyethylene glycol norbornene, polyethylene glycol diacrylate, polylactic acid, derivatives thereof, or combinations thereof.

14. The process of claim 11, wherein the polymerization conditions comprise:
a duration of exposure to the ultraviolet light that is from about 1 millisecond to about 60 seconds;
an energy density of the ultraviolet light that is from about 1 mW/cm² to about 10,000 mW/cm²; or
a combination thereof.

15. The process of claim 14, wherein the duration of exposure to the ultraviolet light is less than about 30 seconds, and the energy density of the ultraviolet light is less than about 1,000 mW/cm².

16. The process of claim 11, wherein a pH of the reaction mixture is from about 5 to about 9.

17. The process of claim 11, wherein:
the dithiol linker has a molecular weight from about 500 Da to about 15,000 Da;
the photoreactive monomer has a number average molecular weight from about 250 Da to about 50,000 Da; or
a combination thereof.

18. The process of claim 11, wherein:
the dithiol linker has a molecular weight from about 1,000 Da to about 5,000 Da; and
the photoreactive monomer has a number average molecular weight from about 15,000 Da to about 35,000 Da.

19. A method, comprising:
introducing a substance that increases or decreases insulin secretion to a composition, the composition comprising:
a hydrogel comprising, in polymerized form, one or more photoreactive monomers and a thiol linker, wherein at least one of the one or more photoreactive monomers comprise a methylene functional group; and
a plurality of beta cells dispersed or encapsulated within the hydrogel; and
monitoring an amount of insulin secretion by at least a portion of the plurality of beta cells.

20. The method of claim 19, wherein the substance that increases or decreases insulin secretion comprises glucose, incretin, acetylcholine, norepinephrine, somatostatin, galanin, prostaglandins, derivatives thereof, mimetics thereof, or combinations thereof.
